SEQUENCE LISTING (27 sequences)

SEQ ID NO: 1 - DNA, 70 bp, Artificial
  Synthetic oligonucleotide primer
cattgccaagccatggactacaaagatgacgatgataaaatcaccaacagcggcgatcggatcaataccg

SEQ ID NO: 2 - DNA, 90 bp, Artificial
  Synthetic oligonucleotide primer
cgctcccgggagctcttattacgcataatccggcacatcatacggataaccgccggtacctgacgcccgcaaggtcggtgacaagaaccg

SEQ ID NO: 3 - DNA, 18 bp, Artificial
  Synthetic oligonucleotide primer
caggcgccattcgccatt

SEQ ID NO: 4 - DNA, 18 bp, Artificial
  Synthetic oligonucleotide primer
ccagctggcgaaaggggg

SEQ ID NO: 5 - DNA, 18 bp, Artificial
  Synthetic oligonucleotide primer
atgtgctgcaaggcgatt

SEQ ID NO: 6 - DNA, 18 bp, Artificial
  Synthetic oligonucleotide primer
gttttcccagtcacgacg

SEQ ID NO: 7 - DNA, 17 bp, Artificial
  Synthetic oligonucleotide primer
gtaaaacgacggccagt

SEQ ID NO: 8 - DNA, 17 bp, Artificial
  Synthetic oligonucleotide primer
gcgttgatgcaatttct

SEQ ID NO: 9 - DNA, 17 bp, Artificial
  Synthetic oligonucleotide primer
cctgctcgcttcgctac

SEQ ID NO: 10 - DNA, 17 bp, Artificial
  Synthetic oligonucleotide primer
ttggagccactatcgac

SEQ ID NO: 11 - DNA, 17 bp, Artificial
  Synthetic oligonucleotide primer
cacacccgtcctgtgga

SEQ ID NO: 12 - DNA, 17 bp, Artificial
  Synthetic oligonucleotide primer
tatccggatatagttcc

SEQ ID NO: 13 - DNA, 20 bp, Artificial
  Synthetic oligonucleotide primer
taatacgactcactataggg

SEQ ID NO: 14 - DNA, 21 bp, Artificial
  Synthetic oligonucleotide primer
accaacagcggcgatcggatc

SEQ ID NO: 15 - DNA, 21 bp, Artificial
  Synthetic oligonucleotide primer
aataccgtgcgcggtcctatc

SEQ ID NO: 16 - DNA, 21 bp, Artificial
  Synthetic oligonucleotide primer
ggatgcccaggagggctgatg

SEQ ID NO: 17 - DNA, 21 bp, Artificial
  Synthetic oligonucleotide primer
cactcgcattatcttctagac

SEQ ID NO: 18 - DNA, 43 bp, Artificial
  Synthetic oligonucleotide primer
aaggttccaacgtctcgcgaccsnnatcgaaagtcgacacatc

SEQ ID NO: 19 - DNA, 40 bp, Artificial
  Synthetic oligonucleotide primer
aaccttggaacgtctcggtcgcgacgtcagtttattggcc

SEQ ID NO: 20 - DNA, 46 bp, Artificial
  Synthetic oligonucleotide primer
aaggttccaacgtctcggtgacsnnsnncgaaaacccgaacagcca

SEQ ID NO: 21 - DNA, 34 bp, Artificial
  Synthetic oligonucleotide primer
aaccttggaacgtctcgtcaccaacatcatggac

SEQ ID NO: 22 - DNA, 46 bp, Artificial
  Synthetic oligonucleotide primer
aaggttccaacgtctccgggtcsnnsnncaagccggtcgccgccac

SEQ ID NO: 23 - DNA, 33 bp, Artificial
  Synthetic oligonucleotide primer
aaccttggacgtctcgacccgccactttcgatg

SEQ ID NO: 24 - protein, 18 aa, Artificial
  Amino Acid Sequence around the OPD gene
MDYKDDDDKITNSGDRIN

SEQ ID NO: 25 - DNA, 56 bp, Artificial
  Vector sequence around the OPD gene
ccatggactacaaagatgacgatgataaaatcaccaacagcggcgatcggatcaat

SEQ ID NO: 26 - protein, 17 aa, Artificial
  Amino Acid Sequence around the OPD Gene
LRASGTGGYPYDVPDYA

SEQ ID NO: 27 - DNA, 63 bp, Artificial
  Sequence around the OPD gene
ttgcgggcgtcaggtaccggcggttatccgtatgatgtgccggattatgcgtaataagagctc

(19) United States
(12) Patent Application Publication
(10) Pub. No.: US 2006/0003347 A1
(43) Pub. Date: Jan. 5, 2006

(54) Single-molecule in vitro evolution

(75) Inventors: Andrew Griffiths, Cambridge (GB); Dan Tawfik, Jerusalem (IL)

Correspondence Address: PALMER & DODGE, LLP;KATHLEEN M. WILLIAMS, 111 HUNTINGTON AVENUE, BOSTON, MA, 02199, US

(73) Assignee: Medical Research Council

(21) Appl. No.: 11/077,956

(22) Filed: Mar. 11, 2005

Related U.S. Application Data
(63) Continuation of application No. PCT/GB03/03924, filed on Sep. 10, 2003.

(30) Foreign Application Priority Data
Sep. 11, 2002 (GB) 0221053.2

(51) Int. Cl. C12Q 1/68

(52) U.S. Cl. 435/6

(57) ABSTRACT

The invention relates to a method for isolating one or more genetic elements encoding a gene product having a desired activity, comprising the steps of: a) providing a population of genetic elements and expressing the genetic elements to produce their respective gene product(s), such that each molecule of gene product is linked to the genetic element encoding it at a ratio of one molecule of gene product per genetic element or less; b) compartmentalising the genetic elements into microcapsules; and c) sorting the genetic elements according to the activity of the gene product.

### RELATED APPLICATIONS

This application is a continuation of International Application No. PCT/GB03/003924, which was filed on 10 Sep. 2003, which designated the United States and was published in English, and which claims the benefit of United Kingdom Application GB0221053.2, filed 11 Sep. 2002. The entire teachings of the above applications are incorporated herein by reference.

### BACKGROUND

The present invention relates to methods for use in in vitro evolution of molecular libraries.

In particular, the present invention relates to methods of selecting nucleic acids encoding gene products according to the activity of the gene product. The invention permits the selection of single molecules of gene product according to activity. In addition, the invention provides highly active phosphotriesterase mutants obtained according to the invention.

Evolution requires the generation of genetic diversity (diversity in nucleic acid) followed by the selection of those nucleic acids which result in beneficial characteristics. Because the nucleic acid and the activity of the encoded gene product of an organism are physically linked (the nucleic acids being confined within the cells which they encode) multiple rounds of mutation and selection can result in the progressive survival of organisms with increasing fitness. Systems for rapid evolution of nucleic acids or proteins in vitro advantageously mimic this process at the molecular level in that the nucleic acid and the activity of the encoded gene product are linked and the activity of the gene product is selectable.

Recent advances in molecular biology have allowed some molecules to be co-selected according to their properties along with the nucleic acids that encode them. The selected nucleic acids can subsequently be cloned for further analysis or use, or subjected to additional rounds of mutation and selection.

Phage display technology has been highly successful as providing a vehicle that allows for the selection of a displayed protein by providing the essential link between nucleic acid and the activity of the encoded gene product (Smith, 1985; Bass et al., 1990; McCafferty et al., 1990; for review see Clackson and Wells, 1994). Filamentous phage particles act as genetic display packages with proteins on the outside and the genetic elements which encode them on the inside. The tight linkage between nucleic acid and the activity of the encoded gene product is a result of the assembly of the phage within bacteria. As individual bacteria are rarely multiply infected, in most cases all the phage produced from an individual bacterium will carry the same genetic element and display the same protein.

However, phage display relies upon the creation of nucleic acid libraries in vivo in bacteria.

Thus, the practical limitation on library size allowed by phage display technology is of the order of 10⁷ to 10¹¹, even taking advantage of λ phage vectors with excisable filamentous phage replicons. The technique has mainly been applied to selection of molecules with binding activity. A small number of proteins with catalytic activity have also been isolated using this technique, however, selection was not directly for the desired catalytic activity, but either for binding to a transition-state analogue (Widersten and Mannervik, 1995) or reaction with a suicide inhibitor (Soumillion et al., 1994; Janda et al., 1997). More recently there have been some examples of enzymes selected using phage-display by product formation (Atwell & Wells, 1999; Demartis et al., 1999; Jestin et al., 1999; Pederson, et al., 1998), but in all these cases selection was not for multiple turnover.

Specific peptide ligands have been selected for binding to receptors by affinity selection using large libraries of peptides linked to the C terminus of the lac repressor LacI (Cull et al., 1992). When expressed in E. coli the repressor protein physically links the ligand to the encoding plasmid by binding to a lac operator sequence on the plasmid.

An entirely in vitro polysome display system has also been reported (Mattheakis et al., 1994; Hanes and Pluckthun, 1997) in which nascent peptides are physically attached via the ribosome to the RNA which encodes them. An alternative, entirely in vitro system for linking genotype to phenotype by making RNA-peptide fusions (Roberts and Szostak, 1997; Nemoto et al., 1997) has also been described.

However, the scope of the above systems is limited to the selection of proteins and furthermore does not allow direct selection for activities other than binding, for example catalytic or regulatory activity.

In vitro RNA selection and evolution (Ellington and Szostak, 1990), sometimes referred to as SELEX (systematic evolution of ligands by exponential enrichment) (Tuerk and Gold, 1990) allows for selection for both binding and chemical activity, but only for nucleic acids. When selection is for binding, a pool of nucleic acids is incubated with immobilised substrate. Non-binders are washed away, then the binders are released, amplified and the whole process is repeated in iterative steps to enrich for better binding sequences. This method can also be adapted to allow isolation of catalytic RNA and DNA (Green and Szostak, 1992; for reviews see Chapman and Szostak, 1994; Joyce, 1994; Gold et al., 1995; Moore, 1995).

However, selection for “catalytic” or binding activity using SELEX is only possible because the same molecule performs the dual role of carrying the genetic information and being the catalyst or binding molecule (aptamer). When selection is for “auto-catalysis” the same molecule must also perform the third role of being a substrate. Since the genetic element must play the role of both the substrate and the catalyst, selection is only possible for single turnover events. Because the “catalyst” is in this process itself modified, it is by definition not a true catalyst. Additionally, proteins may not be selected using the SELEX procedure. The range of catalysts, substrates and reactions which can be selected is therefore severely limited.

Those of the above methods that allow for iterative rounds of mutation and selection are mimicking in vitro mechanisms usually ascribed to the process of evolution: iterative variation, progressive selection for a desired the activity and replication. However, none of the methods so far developed have provided molecules of comparable diversity and functional efficacy to those that are found naturally. Additionally, there are no man-made “evolution” systems which can evolve both nucleic acids and proteins to effect the full range of biochemical and biological activities (for example, binding, catalytic and regulatory activities) and that can combine several processes leading to a desired product or activity.

In Tawfik and Griffiths (1998), and in International patent application WO 99/02671, we describe a system for in vitro evolution that overcomes many of the limitations described above by using compartmentalisation in microcapsules to link genotype and phenotype at the molecular level.

In Tawfik and Griffiths (1998), and in several embodiments of International patent application WO 99/02671, the desired activity of a gene product results in a modification of the genetic element which encoded it (and is present in the same microcapsule). The modified genetic element can then be selected in a subsequent step.

In WO 00/40712 a variant of this technique is described, in which the modification of the genetic element causes a change in the optical properties of the element itself.

### SUMMARY OF THE INVENTION

We describe herein a novel technique for the selection of genes and gene products according to the activity of the gene product at the single molecule level. In a first aspect, therefore, there is provided a method for isolating one or more genetic elements encoding a gene product having a desired activity, comprising the steps of:
a) providing a population of genetic elements and expressing the genetic elements to produce their respective gene product(s), such that each molecule of gene product is linked to the genetic element encoding it at a ratio of one molecule of gene product per genetic element or less;
b) compartmentalising the genetic elements into microcapsules; and
c) sorting the genetic elements according to the activity of the gene product.

Mimicking nature by applying directed evolution in the laboratory is a very powerful strategy (Georgiou, 2000; Griffiths and Tawfik, 2000; Ness et al., 2000; Petrounia and Arnold, 2000; Pluckthun et al., 2000; Soumillion and Fastrez, 2001; Wahler and Reymond, 2001). Both natural and directed evolution require a link between genotype (a nucleic acid that can be replicated) and phenotype (a functional trait such as binding or catalytic activity) (Griffiths and Tawfik, 2000). In vitro, this linkage is usually achieved by physically linking genes to the proteins they encode by a variety of techniques, including display on phage, viruses, bacteria and yeast, plasmid-display, ribosome-display and mRNA-peptide fusion. These ‘display technologies’, have proven highly successful in the generation of binding proteins (Amstutz et al., 2001; Georgiou et al., 1997; Griffiths and Duncan, 1998; Keefe and Szostak, 2001; Pluckthun et al., 2000; Schatz et al., 1996; Sidhu, 2000; Wittrup, 2001).

In contrast, selection of enzymes by display approaches has met with little success. Indirect selections—by binding to transition state analogues or enzyme inhibitors—have generally failed to produce potent catalysts (Griffiths and Tawfik, 2000). Single-turnover, intramolecular selections of enzymes displayed on phage were demonstrated but these impose severe limitations (Atwell and Wells, 1999; Griffiths and Tawfik, 2000). To evolve proficient enzymes, the selection (or screen) should be simultaneous and direct for all enzymatic properties: substrate recognition, formation of a specific product, rate acceleration and turnover (the ability of a single active-site to catalyse the conversion of numerous substrate molecules). The only efficient selection for turnover described is for variants of the E. coli outer membrane protein, OmpT, using a positively charged fluorogenic substrate which binds to the negatively charged surface of E. coli allowing them to be sorted by flow cytometry (Olsen et al., 2000).

Direct selection for all enzymatic properties can be achieved by compartmentalisation in cells (as in nature), typically by screening 10³-10⁵ clones in a plate assay using a fluorogenic or chromogenic substrate. However, crossing long evolutionary distances, and evolving completely novel proteins and activities, requires much larger libraries (Griffiths and Tawfik, 2000; Keefe and Szostak, 2001). In these cases, selection rather than screening is preferable.

Unfortunately, in vivo selections are usually (but not always (Firestine et al., 2000)) restricted to functions that affect the viability of the organism and are often complicated by the complex intracellular environment and the need to transform the gene-library. There is little doubt therefore, that purely in vitro systems will eventually prove advantageous (Fastrez, 1997; Minshull and Stemmer, 1999; Pluckthun et al., 2000).

As used herein, a genetic element is a molecule or molecular construct comprising a nucleic acid. The genetic elements of the present invention may comprise any nucleic acid (for example, DNA, RNA or any analogue, natural or artificial, thereof). The nucleic acid component of the genetic element may moreover be linked, covalently or non-covalently, to one or more molecules or structures, including proteins, chemical entities and groups, and solid-phase supports such as beads (including nonmagnetic, magnetic and paramagnetic beads), and the like. In the method of the invention, these structures or molecules are designed to assist in the sorting and/or isolation of the genetic element encoding a gene product with the desired activity.

Expression, as used herein, is used in its broadest meaning, to signify that a nucleic acid contained in the genetic element is converted into its gene product. Thus, where the nucleic acid is DNA, expression refers to the transcription of the DNA into RNA; where this RNA codes for protein, expression may also refer to the translation of the RNA into protein. Where the nucleic acid is RNA, expression may refer to the replication of this RNA into further RNA copies, the reverse transcription of the RNA into DNA and optionally the transcription of this DNA into further RNA molecule(s), as well as optionally the translation of any of the RNA species produced into protein. Preferably, therefore, expression is performed by one or more processes selected from the group consisting of transcription, reverse transcription, replication and translation.

Expression of the genetic element may thus be directed into either DNA, RNA or protein, or a nucleic acid or protein containing unnatural bases or amino acids (the gene product) within the microcapsule of the invention, so that the gene product is confined within the same microcapsule as the genetic element.

The genetic element and the gene product thereby encoded are linked by confining each genetic element and the respective gene product encoded by the genetic element within the same microcapsule. In this way the gene product in one microcapsule cannot interact with genetic elements in any other microcapsules. Further linking means are employed to link gene products to the genetic elements encoding them, as set forth below.

The term “microcapsule” is used herein in accordance with the meaning normally assigned thereto in the art and further described hereinbelow. In essence, however, a microcapsule is an artificial compartment whose delimiting borders restrict the exchange of the components of the molecular mechanisms described herein which allow the sorting of the genetic elements according to the function of the gene products which they encode.

Preferably, the microcapsules used in the method of the present invention are capable of being produced in very large numbers, and thereby to compartmentalise a library of genetic elements which encodes a repertoire of gene products.

The genetic elements are sorted by a multi-step procedure, which involves at least two steps, for example, in order to allow the exposure of the genetic elements to conditions which permit at least two separate reactions to occur. As will be apparent to persons skilled in the art, the first step of the invention advantageously results in conditions which permit the expression of the genetic elements—be it transcription, transcription and/or translation, replication or the like. Under these conditions, it may not be possible to select for a particular gene product activity, for example because the gene product may not be active under these conditions, or because the expression system contains an interfering activity.

The invention therefore provides a method comprising linking the gene products to the genetic elements encoding them and isolating the complexes thereby formed. This allows for the genetic elements and their associated gene products to be isolated from the capsules before sorting according to gene product activity takes place. In a preferred embodiment, the complexes are subjected to a compartmentalisation step prior to isolating the genetic elements encoding a gene product having the desired activity, although where compartmentalisation is used in the expression step, the sorting for activity may take place in the same compartments. This compartmentalisation step, which advantageously takes place in microcapsules, permits the performance of further reactions, under different conditions, in an environment where the genetic elements and their respective gene products are physically linked.

The “secondary encapsulation” may be performed with genetic elements linked to gene products by means other than encapsulation, such as by phage display, polysome display, RNA-peptide fusion or lac repressor peptide fusion.

Preferably, the genetic element/gene product complexes are produced by microencapsulation. Thus, the invention provides, in a second aspect, a method for isolating one or more genetic elements encoding a gene product having a desired activity, comprising the steps of:
(a) compartmentalising genetic elements into microcapsules;
(b) expressing the genetic elements to produce their respective gene products within the microcapsules;
(c) linking the gene products to the genetic elements at a ratio of one molecule of gene product per genetic element or less; and
(d) sorting the genetic elements according to the activity of the gene product.

Advantageously, step (d) is carried out according to the first aspect of the invention. Preferably, the genetic elements are pooled subsequent to linkage to the gene product, optionally subjected to selection for expression of the gene product and recompartmentalised for sorting according to activity of the gene product. Importantly, the ratio of gene product to genetic element is one or less, arranged such that substantially each genetic element is linked to only a single molecule of gene product.

The selected genetic element(s) may also be subjected to subsequent, optionally more stringent rounds of sorting in iteratively repeated steps, reapplying the method of the invention either in its entirety or in selected steps only. By tailoring the conditions appropriately, genetic elements encoding gene products having a better optimised activity may be isolated after each round of selection.

Additionally, the genetic elements isolated after a first round of sorting may be subjected to mutagenesis before repeating the sorting by iterative repetition of the steps of the method of the invention as set out above. After each round of mutagenesis, some genetic elements will have been modified in such a way that the activity of the gene products is enhanced.

Moreover, the nucleic acid in the selected genetic elements can be cloned into an expression vector to allow further characterisation of the genetic elements and their products.

In a third aspect, the invention provides a product when selected according to the first or second aspect of the invention. As used in this context, a “product” may refer to a gene product, selectable according to the invention, or the genetic element (or genetic information comprised therein).

In an advantageous embodiment, the product has increased activity over a wild-type or pre-existing equivalent. Advantageously, the product has an activity superior to any known pre-existing equivalent. For example, where the product is an enzyme, the k_{cat} is advantageously higher than any previously known for a molecule with the same enzymatic specificity.

Advantageously, the k_{cat} is 10× or more greater than any previously known, preferably 25× or more, preferably 50× or more and more preferably 100× or more. It can advantageously by 123× greater.

In a particular embodiment, the enzyme is a mutant of a phosphotriesterase which has a higher k_{cat} than any phosphotriesterase of the prior art. Advantageously, the k_{cat} is k_{cat} of 10⁵ s⁻¹ or more, preferably 2.8×10⁵s⁻¹.

Advantageously, the phosphotriesterase of the invention comprises one or more of the mutations selected from the following groups:
I106T and F132L;
I106S, F1332L, S308L and Y309R;
I106S;
I106D, W131Y and F132S;
I106L.

In a third aspect, the invention provides a method for preparing a gene product, comprising the steps of:
(a) preparing a genetic element encoding the gene product;
(b) compartmentalising genetic elements into microcapsules;
(c) expressing the genetic elements to produce their respective gene products within the microcapsules;
(d) linking the gene products to the genetic elements such that each genetic element is linked to no more than one molecule of its gene product;
(e) within microcapsules, sorting the genetic elements which produce the gene product(s) having the desired activity; and
(f) expressing the gene product having the desired activity.

In accordance with the third aspect, step (a) preferably comprises preparing a repertoire of genetic elements, wherein each genetic element encodes a potentially differing gene product.

Repertoires may be generated by conventional techniques, such as those employed for the generation of libraries intended for selection by methods such as phage display. Gene products having the desired activity may be selected from the repertoire, according to the present invention.

In a fourth aspect, the invention provides a method for screening a compound or compounds capable of modulation the activity of a gene product, comprising the steps of:
(a) preparing a repertoire of genetic elements encoding gene product;
(b) compartmentalising genetic elements into microcapsules;
(c) expressing the genetic elements to produce their respective gene products within the microcapsules;
(d) linking the gene products to the genetic elements such that each genetic element is linked to no more than one molecule of its gene product;
(e) within microcapsules, sorting the genetic elements which produce the gene product(s) having the desired activity; and
(f) contacting a gene product having the desired activity with the compound or compounds and monitoring the modulation of an activity of the gene product by the compound or compounds.

Advantageously, the method further comprises the step of:
(g) identifying the compound or compounds capable of modulating the activity of the gene product and synthesising said compound or compounds.

This selection system can be configured to select for RNA, DNA or protein molecules with catalytic, regulatory or other activities.

### BRIEF DESCRIPTION OF THE FIGURES

FIG. 1. Creation of microbead-display libraries and selection for catalysis by compartmentalisation. (A) The creation of microbead-display libraries. A repertoire of genes encoding protein variants, each with a common N- or C-terminal epitope tag, are linked to streptavidin-coated beads carrying antibodies that bind the epitope tag at, on average, less than one gene per bead (1). The beads are compartmentalised in a water-in-oil emulsion to give, on average, less than one bead per compartment (2), and transcribed and translated in vitro in the compartments. Consequently, in each compartment, the translated protein (10-100 copies) becomes attached to the gene that encodes it via the bead (3). The emulsion is broken (4), and the microbeads carrying the display-library isolated (5). (B) Enzyme selection by compartmentalisation. Microbead-display libraries are compartmentalised in a water-in-oil emulsion (1) and a soluble substrate attached to caged-biotin is added. The substrate is converted to product only in compartments containing beads displaying active enzymes (2). The emulsion is then irradiated to uncage the biotin (3).

Consequently, in a compartment containing a gene encoding an enzyme, the product becomes attached to the gene via the bead (4). In other compartments, in which the genes do not encode an enzyme for the selected reaction, the intact substrate becomes attached to the gene. The emulsion is broken (5), and the beads incubated with anti-product antibodies (6). Product-coated beads can then be enriched (together with the genes attached to them) either by affinity purification or, after reacting with a fluorescently labelled antibody, by flow cytometry.

FIG. 2. The pIVEX-OPD vector and the annealing sites of the oligonucleotide primers used for PCR amplification. (A) Schematic representation of the region of the vector pIVEX-OPD around the cloned OPD gene. The NcoI and SacI restriction sites used for cloning and the translated open reading frame (Translated ORF) which encodes PTE (encoded by the OPD gene[Mulbry, 1989]) with N-terminal Flag[Chiang, 1993 #91] and C-terminal HA[Field, 1988] epitope tags are indicated. The region of the OPD gene deleted in pIVEX-ΔOPD is also shown. The vector contains a T7 promoter, enhancer, terminator and ribosome binding site (rbs) for efficient expression in vitro. The annealing sites for oligonucleotide primers used for PCR and listed in Table 1 are indicated (a to J). (B) The sequence of pIVEX-OPD between the NcoI and SacI sites. The sequence outside this region is as pIVEX2.2b Nde (Roche). The sequences encoding the Flag and HA epitope tags are indicated and OPD gene sequence [Mulbry, 1989] is in bold italics.

FIG. 3. Phosphotriesterase (PTE) substrates. (A) PTE catalysed hydrolysis of paraoxon. (B) For selection, the PTE substrate paraoxon was modified by substituting one of its ethyl groups with a linker connected to caged-biotin [Pirrung, 1996 #61]. PTE-catalysed hydrolysis of the resulting substrate (EtNP-cgB) gives p-nitrophenol and the corresponding phosphodiester Et-cgB. Irradiation at 354 nm releases the caging group and carbon dioxide to yield the (uncaged) biotinylated substrate (EtNP-B) or product (Et-B). (C) An alternative PTE substrate for selection, EtNP-ATFB was created by using a photolabelling group 4-azido-2,3,5,6-tetrafluoro benzoic acid in place of caged-biotin.

FIG. 4. Detection of substrate and product on microbeads by flow cytometry.

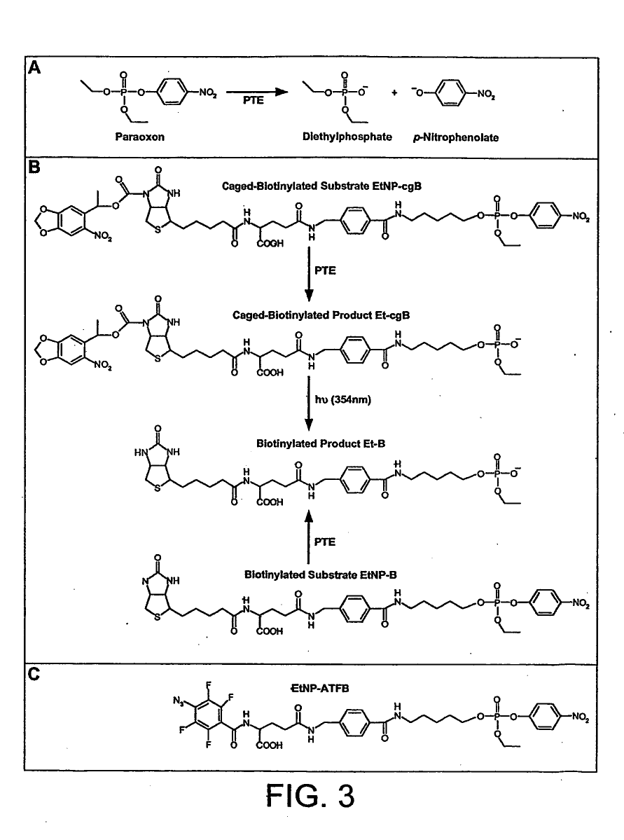

Streptavidin-coated beads were coated with biotinylated anti-HA antibodies and then with mixtures of the biotinylated PTE substrate EtNP-B and the biotinylated product EtNP-B (FIG. 3B). After fluorescent labelling using anti-product antibodies, the beads were analysed by flow cytometry. The levels of fluorescence (FL1-H) on single, unagregated beads (gated using forward- and side-scatter as in FIG. 5) are plotted as histograms and shown for beads coated with 0% product (100% substrate), 5% product (95% substrate), 12.5% product (87.5% substrate), 25% product (75% substrate) and 50% product (50% substrate).

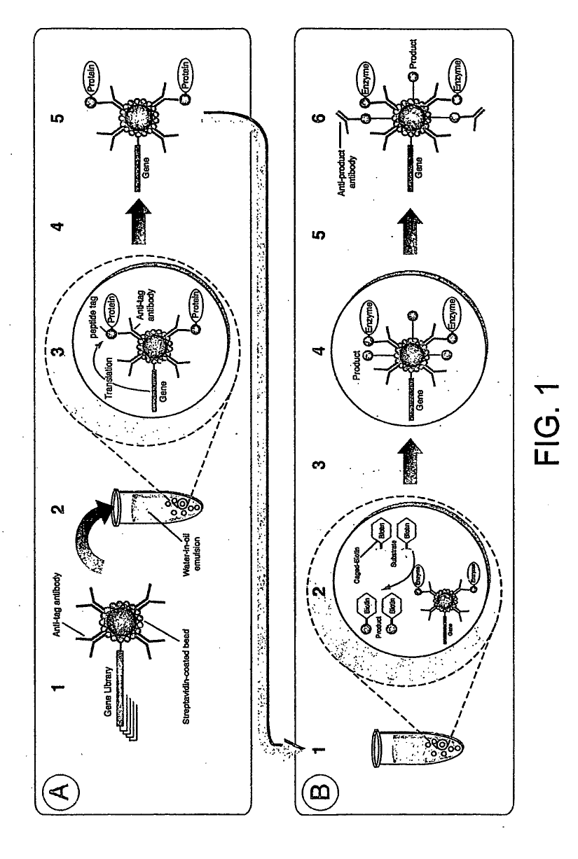

FIG. 5. Selections for genes encoding PTE. Microbeads displaying the proteins encoded by the genes attached to them (FIG. 1A) were created using the OPD and ΔOPD genes and mixtures thereof (Table 1). These were then selected for enzymatic activity (FIG. 1B) using EtNP-cgB (FIG. 3B) as substrate. After fluorescent labelling using anti-product antibodies the beads were analysed by flow cytometry. Forward-scatter (FSC-H) and side-scatter (SSC-H) indicated that most of the beads were single and unagregated (95% of total events were in R1 of the dot-plot, column 2). The levels of fluorescence (FL1-H) on single, unsorted beads (gated through R1) are plotted as histograms (column 1). The ‘positive’, highly fluorescent beads (in region M1) were sorted from ‘negative’, low fluorescence beads and re-analysed (column 2). The genes on the sorted ‘positive’ beads (and on the unsorted bead mixture) were PCR-amplified and the resulting DNA analysed by gel electrophoresis (column 3). The OPD and ΔOPD genes gave rise to bands of 697 bp and 439 bp respectively. Markers, φX174-HaeIII digest.

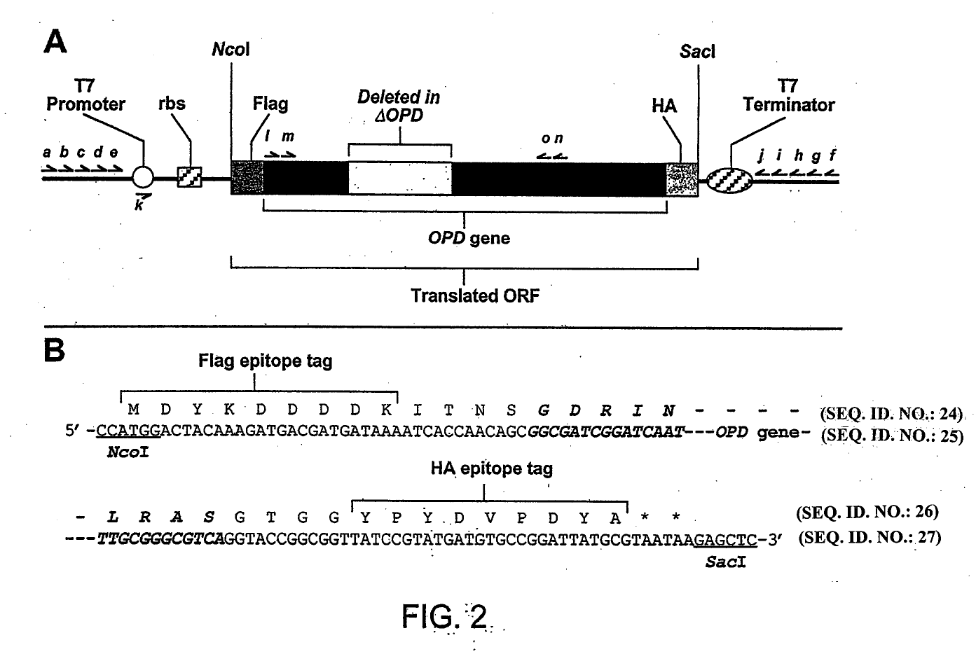

FIG. 6. The identification of single PTE molecules. Recombinant, epitope-tagged PTE (FIG. 2) was bound to streptavidin-coated beads via the N-Flag tag at different stoichiometric ratios as indicated. The beads were selected for catalysis as above (FIG. 1B) using EtNP-cgB (FIG. 3B) as substrate, either compartmentalised (in an emulsion), or non-compartmentalised. After fluorescent labelling using antibody-product antibodies, the beads were analysed by flow cytometry. The levels of fluorescence (FL1-H) on single, unagregated beads (gated using forward- and side-scatter as in FIG. 5) are shown for non-compartmentalised (column 1) and compartmentalised (emulsified) beads (column 2).

FIG. 7. Graphic representation of the substrate binding pockets of PTE. Panels A and B are based on the co-ordinates of zinc-containing PTE with the bound substrate analogue diethyl 4-methylbenzylphosphate[Vanhooke, 1996]. Panel A shows the amino-acid residues whose side chains define the substrate binding site. Residues forming the small subsite are annotated in yellow, those forming the large subsite in red and those forming the leaving group subsite in white. Panel B shows the five amino acid residues randomised in the libraries.

FIG. 8. Selection of PTE libraries. PTE libraries were selected for phosphotriesterase activity using EtNP-cgB (FIG. 3B) as substrate as described in FIG. 1. After fluorescent labelling using anti-product antibodies the beads were analysed and sorted by flow cytometry. The levels of fluorescence (FL1-H) on single, unsorted beads (gated using forward- and side-scatter as in FIG. 5) in each round of selection (rows b to g) are plotted as histograms, along with the results for beads not coated with DNA (row a). Results are shown for the selection of Library B (column 1), Library C (column 2) and Library D (column 3). In all except the final round of selection a single gate (M1), set to include only 1% of beads which were not coated with DNA (row a), was used to sort high fluorescence beads. In the final round of selection three gates (M1, M2 and M3) were used to sort Libraries B and D and two gates (M1 and M2) were used to sort library C. The beads sorted through these gates were re-analysed and are shown as unfilled histograms. In addition, the DNA on beads sorted through each of these gates, together with unselected DNA and DNA from all previous rounds of selection, was amplified, translated in vitro, incubated with Zn²⁺ to assemble the PTE metalloenzyme and phosphotriesterase activity measured using paraoxon (FIG. 3A) as substrate. Catalytic activities are expressed as percentage of the activity from in vitro translation of an identical number of wild-type OPD genes and are indicated as annotations on the histograms of the sorted beads from the final round of selection and plotted for all rounds of selection in Panel 4.

FIG. 9. Eadie-Hofstee plot of kinetic data for wild-type PTE and mutant H5.

### DETAILED DESCRIPTION OF THE INVENTION

(A) General Description

The microcapsules of the present invention require appropriate physical properties to allow the working of the invention.

First, to ensure that the genetic elements and gene products may not diffuse between microcapsules, the contents of each microcapsule are preferably isolated from the contents of the surrounding microcapsules, so that there is no or little exchange of the genetic elements and gene products between the microcapsules over the timescale of the experiment.

Second, the method of the present invention requires that there are only a limited number of genetic elements per microcapsule. This ensures that the gene product of an individual genetic element will be isolated from other genetic elements. Thus, coupling between genetic element and gene product will be highly specific. The enrichment factor is greatest with on average one or fewer genetic elements per microcapsule, the linkage between nucleic acid and the activity of the encoded gene product being as tight as is possible, since the gene product of an individual genetic element will be isolated from the products of all other genetic elements. However, even if the theoretically optimal situation of, on average, a single genetic element or less per microcapsule is not used, a ratio of 5, 10, 50, 100 or 1000 or more genetic elements per microcapsule may prove beneficial in sorting a large library. Subsequent rounds of sorting, including, renewed encapsulation with differing genetic element distribution, will permit more stringent sorting of the genetic elements. Preferably, there is a single genetic element, or fewer, per microcapsule.

Third, the formation and the composition of the microcapsules advantageously does not abolish the function of the machinery the expression of the genetic elements and the activity of the gene products.

The appropriate system(s) may vary depending on the precise nature of the requirements in each application of the invention, as will be apparent to the skilled person.

A wide variety of microencapsulation procedures are available (see Benita, 1996) and may be used to create the microcapsules used in accordance with the present invention. Indeed, more than 200 microencapsulation methods have been identified in the literature (Finch, 1993).

These include membrane enveloped aqueous vesicles such as lipid vesicles (liposomes) (New, 1990) and non-ionic surfactant vesicles (van Hal et al., 1996). These are closed-membranous capsules of single or multiple bilayers of non-covalently assembled molecules, with each bilayer separated from its neighbour by an aqueous compartment. In the case of liposomes the membrane is composed of lipid molecules; these are usually phospholipids but sterols such as cholesterol may also be incorporated into the membranes (New, 1990). A variety of enzyme-catalysed biochemical reactions, including RNA and DNA polymerisation, can be performed within liposomes (Chakrabarti et al., 1994; Oberholzer et al., 1995a; Oberholzer et al., 1995b; Walde et al., 1994; Wick & Luisi, 1996).

With a membrane-enveloped vesicle system much of the aqueous phase is outside the vesicles and is therefore non-compartmentalised. This continuous, aqueous phase is removed or the biological systems in it inhibited or destroyed (for example, by digestion of nucleic acids with DNase or RNase) in order that the reactions are limited to the microcapsules (Luisi et al., 1987).

Enzyme-catalysed biochemical reactions have also been demonstrated in microcapsules generated by a variety of other methods. Many enzymes are active in reverse micellar solutions (Bru & Walde, 1991; Bru & Walde, 1993; Creagh et al., 1993; Haber et al., 1993; Kumar et al., 1989; Luisi & B., 1987; Mao & Walde, 1991; Mao et al., 1992; Perez et al., 1992; Walde et al., 1994; Walde et al., 1993; Walde et al., 1988) such as the AOT-isooctane-water system (Menger & Yamada, 1979).

Microcapsules can also be generated by interfacial polymerisation and interfacial complexation (Whateley, 1996). Microcapsules of this sort can have rigid, nonpermeable membranes, or semipermeable membranes. Semipermeable microcapsules bordered by cellulose nitrate membranes, polyamide membranes and lipid-polyamide membranes can all support biochemical reactions, including multienzyme systems (Chang, 1987; Chang, 1992; Lim, 1984). Alginate/polylysine microcapsules (Lim & Sun, 1980), which can be formed under very mild conditions, have also proven to be very biocompatible, providing, for example, an effective method of encapsulating living cells and tissues (Chang, 1992; Sun et al., 1992).

Non-membranous microencapsulation systems based on phase partitioning of an aqueous environment in a colloidal system, such as an emulsion, may also be used.

Preferably, the microcapsules of the present invention are formed from emulsions; heterogeneous systems of two immiscible liquid phases with one of the phases dispersed in the other as droplets of microscopic or colloidal size (Becher, 1957; Sherman, 1968; Lissant, 1974; Lissant, 1984).

Emulsions may be produced from any suitable combination of immiscible liquids. Preferably the emulsion of the present invention has water (containing the biochemical components) as the phase present in the form of finely divided droplets (the disperse, internal or discontinuous phase) and a hydrophobic, immiscible liquid (an oil') as the matrix in which these droplets are suspended (the nondisperse, continuous or external phase). Such emulsions are termed “water-in-oil” (W/O). This has the advantage that the entire aqueous phase containing the biochemical components is compartmentalised in discreet droplets (the internal phase). The external phase, being a hydrophobic oil, generally contains none of the biochemical components and hence is inert.

The emulsion may be stabilised by addition of one or more surface-active agents (surfactants). These surfactants are termed emulsifying agents and act at the water/oil interface to prevent (or at least delay) separation of the phases. Many oils and many emulsifiers can be used for the generation of water-in-oil emulsions; a recent compilation listed over 16,000 surfactants, many of which are used as emulsifying agents (Ash and Ash, 1993). Suitable oils include light white mineral oil and non-ionic surfactants (Schick, 1966) such as sorbitan monooleate (Span™ 80; ICI) and polyoxyethylenesorbitan monooleate (Tween™ 80; ICI).

The use of anionic surfactants may also be beneficial. Suitable surfactants include sodium cholate and sodium taurocholate. Particularly preferred is sodium deoxycholate, preferably at a concentration of 0.5% w/v, or below. Inclusion of such surfactants can in some cases increase the expression of the genetic elements and/or the activity of the gene products. Addition of some anionic surfactants to a non-emulsified reaction mixture completely abolishes translation. During emulsification, however, the surfactant is transferred from the aqueous phase into the interface and activity is restored. Addition of an anionic surfactant to the mixtures to be emulsified ensures that reactions proceed only after compartmentalisation.

Creation of an emulsion generally requires the application of mechanical energy to force the phases together. There are a variety of ways of doing this which utilise a variety of mechanical devices, including stirrers (such as magnetic stir-bars, propeller and turbine stirrers, paddle devices and whisks), homogenisers (including rotor-stator homogenisers, high-pressure valve homogenisers and jet homogenisers), colloid mills, ultrasound and ‘membrane emulsification’ devices (Becher, 1957; Dickinson, 1994).

Aqueous microcapsules formed in water-in-oil emulsions are generally stable with little if any exchange of genetic elements or gene products between microcapsules. Additionally, we have demonstrated that several biochemical reactions proceed in emulsion microcapsules.

Moreover, complicated biochemical processes, notably gene transcription and translation are also active in emulsion microcapsules. The technology exists to create emulsions with volumes all the way up to industrial scales of thousands of litres (Becher, 1957; Sherman, 1968; Lissant, 1974; Lissant, 1984).

The preferred microcapsule size will vary depending upon the precise requirements of any individual selection process that is to be performed according to the present invention. In all cases, there will be an optimal balance between gene library size, the required enrichment and the required concentration of components in the individual microcapsules to achieve efficient expression and reactivity of the gene products.

The processes of expression occurs within each individual microcapsule provided by the present invention. Both in vitro transcription and coupled transcription-translation become less efficient at sub-nanomolar DNA concentrations. Because of the requirement for only a limited number of DNA molecules to be present in each microcapsule, this therefore sets a practical upper limit on the possible microcapsule size. Preferably, the mean volume of the microcapsules is less that 5.2×10⁻¹⁶ m³, (corresponding to a spherical microcapsule of diameter less than 10 μm, more preferably less than 6.5×10⁻¹⁷ m³ (5 μm diameter), more preferably about 4.2×10⁻¹⁸ m³ (2 μm diameter) and ideally about 9×10⁻¹⁸ m³ (2.6 μm diameter).

The effective DNA or RNA concentration in the microcapsules may be artificially increased by various methods that will be well-known to those versed in the art. These include, for example, the addition of volume excluding chemicals such as polyethylene glycols (PEG) and a variety of gene amplification techniques, including transcription using RNA polymerases including those from bacteria such as E. coli (Roberts, 1969; Blattner and Dahlberg, 1972; Roberts et al., 1975; Rosenberg et al., 1975), eukaryotes e.g. (Weil et al., 1979; Manley et al., 1983) and bacteriophage such as T7, T3 and SP6 (Melton et al., 1984); the polymerase chain reaction (PCR) (Saiki et al., 1988); Qb replicase amplification (Miele et al., 1983; Cahill et al., 1991; Chetverin and Spirin, 1995; Katanaev et al., 1995); the ligase chain reaction (LCR) (Landegren et al., 1988; Barany, 1991); and self-sustained sequence replication system (Fahy et al., 1991) and strand displacement amplification (Walker et al., 1992). Gene amplification techniques requiring thermal cycling such as PCR and LCR may be used if the emulsions and the in vitro transcription or coupled transcription-translation systems are thermostable (for example, the coupled transcription-translation systems can be made from a thermostable organism such as Thermus aquaticus).

Increasing the effective local nucleic acid concentration enables larger microcapsules to be used effectively. This allows a preferred practical upper limit to the microcapsule volume of about 5.2×10⁻¹⁶ m³ (corresponding to a sphere of diameter 10 μm).

The microcapsule size is preferably sufficiently large to accommodate all of the required components of the biochemical reactions that are needed to occur within the microcapsule.

For example, in vitro, both transcription reactions and coupled transcription-translation reactions require a total nucleoside triphosphate concentration of about 2 mM.

For example, in order to transcribe a gene to a single short RNA molecule of 500 bases in length, this would require a minimum of 500 molecules of nucleoside triphosphate per microcapsule (8.33×10⁻²² moles). In order to constitute a 2 mM solution, this number of molecules is contained within a microcapsule of volume 4.17×10⁻¹⁹ litres (4.17×10⁻²² m³ which if spherical would have a diameter of 93 nm.

Furthermore, particularly in the case of reactions involving translation, it is to be noted that the ribosomes necessary for the translation to occur are themselves approximately 20 nm in diameter. Hence, the preferred lower limit for microcapsules is a diameter of approximately 0.1 μm (100 nm).

Therefore, the microcapsule volume is preferably of the order of between 5.2×10⁻²² m³ and 5.2×10⁻¹⁶ m³ corresponding to a sphere of diameter between 0.1 μm and 10 μm, more preferably of between about 5.2×10⁻¹⁹ m3 and 6.5×10⁻¹⁷ m³ (1 μm and 5 μm). Sphere diameters of about 2.6 μm are most advantageous.

It is no coincidence that the preferred dimensions of the compartments (droplets of 2.6 μm mean diameter) closely resemble those of bacteria, for example, Escherichia are 1.1-1.5×2.0-6.0 μm rods and Azotobacter are 1.5-2.0 μm diameter ovoid cells. In its simplest form, Darwinian evolution is based on a ‘one genotype one phenotype’ mechanism. The concentration of a single compartmentalised gene, or genome, drops from 0.4 nM in a compartment of 2 μm diameter, to 25 pM in a compartment of 5 μm diameter. The prokaryotic transcription/translation machinery has evolved to operate in compartments of ˜1-2 μm diameter, where single genes are at approximately nanomolar concentrations. A single gene, in a compartment of 2.6 μm diameter is at a concentration of 0.2 nM. This gene concentration is high enough for efficient translation. Compartmentalisation in such a volume also ensures that even if only a single molecule of the gene product is formed it is present at about 0.2 nM, which is important if the gene product is to have a modifying activity of the genetic element itself. The volume of the microcapsule is thus selected bearing in mind not only the requirements for transcription and translation of the genetic element, but also the modifying activity required of the gene product in the method of the invention.

The size of emulsion microcapsules may be varied simply by tailoring the emulsion conditions used to form the emulsion according to requirements of the selection system. The larger the microcapsule size, the larger is the volume that will be required to encapsulate a given genetic element library, since the ultimately limiting factor will be the size of the microcapsule and thus the number of microcapsules possible per unit volume.

The size of the microcapsules is selected not only having regard to the requirements of the transcription/translation system, but also those of the selection system employed for the genetic element. Thus, the components of the selection system, such as a chemical modification system, may require reaction volumes and/or reagent concentrations which are not optimal for transcription/translation. As set forth herein, such requirements may be accommodated by a secondary re-encapsulation step; moreover, they may be accommodated by selecting the microcapsule size in order to maximise transcription/translation and selection as a whole. Empirical determination of optimal microcapsule volume and reagent concentration, for example as set forth herein, is preferred.

A “genetic element” in accordance with the present invention is as described above. Preferably, a genetic element is a molecule or construct selected from the group consisting of a DNA molecule, an RNA molecule, a partially or wholly artificial nucleic acid molecule consisting of exclusively synthetic or a mixture of naturally-occurring and synthetic bases, any one of the foregoing linked to a polypeptide, and any one of the foregoing linked to any other molecular group or construct. Advantageously, the other molecular group or construct may be selected from the group consisting of nucleic acids, polymeric substances, particularly beads, for example polystyrene beads, and magnetic or paramagnetic substances such as magnetic or paramagnetic beads.

The nucleic acid portion of the genetic element may comprise suitable regulatory sequences, such as those required for efficient expression of the gene product, for example promoters, enhancers, translational initiation sequences, polyadenylation sequences, splice sites and the like.

As will be apparent from the following, in many cases the polypeptide or other molecular group or construct is a ligand or a substrate which directly or indirectly binds to or reacts with the gene product in order to link the genetic element to the gene product. This allows the sorting of the genetic element on the basis of the activity of the gene product in a subsequent selection procedure. The ligand or substrate can be connected to the nucleic acid by a variety of means that will be apparent to those skilled in the art (see, for example, Hermanson, 1996).

One way in which the nucleic acid molecule may be linked to a ligand or substrate is through biotinylation. This can be done by PCR amplification with a 5′-biotinylation primer such that the biotin and nucleic acid are covalently linked.

The ligand or substrate can be attached to the modified nucleic acid by a variety of means that will be apparent to those of skill in the art (see, for example, Hermanson, 1996). A biotinylated nucleic acid may be coupled to a polystyrene or paramagnetic microbead (0.02 to approx. 5.0 μm in diameter) that is coated with avidin or streptavidin, that will therefore bind the nucleic acid with very high affinity. This bead can be derivatised with substrate or ligand by any suitable method such as by adding biotinylated substrate or by covalent coupling.

Alternatively, a biotinylated nucleic acid may be coupled to avidin or streptavidin complexed to a large protein molecule such as thyroglobulin (669 Kd) or ferritin (440 Kd). This complex can be derivatised with substrate or ligand, for example by covalent coupling to the ε-amino group of lysines or through a non-covalent interaction such as biotin-avidin.

The substrate may be present in a form unlinked to the genetic element but containing an inactive “tag” that requires a further step to activate it such as photoactivation (e.g. of a “caged” biotin analogue, (Sundberg et al., 1995; Pirrung and Huang, 1996)). The catalyst to be selected then converts the substrate to product. The “tag” is then activated and the “tagged” substrate and/or product bound by a tag-binding molecule (e.g. avidin or streptavidin) complexed with the nucleic acid. The ratio of substrate to product attached to the nucleic acid via the “tag” will therefore reflect the ratio of the substrate and product in solution.

An alternative is to couple the nucleic acid to a product-specific antibody (or other product-specific molecule). In this scenario, the substrate (or one of the substrates) is present in each microcapsule unlinked to the genetic element, but has a molecular “tag” (for example biotin, DIG or DNP or a fluorescent group). When the catalyst to be selected converts the substrate to product, the product retains the “tag” and is then captured in the microcapsule by the product-specific antibody. In this way the genetic element only becomes associated with the “tag” when it encodes or produces an enzyme capable of converting substrate to product.

The terms “isolating”, “sorting” and “selecting”, as well as variations thereof, are used herein. Isolation, according to the present invention, refers to the process of separating an entity from a heterogeneous population, for example a mixture, such that it is free of at least one substance with which it was associated before the isolation process. In a preferred embodiment, isolation refers to purification of an entity essentially to homogeneity. Sorting of an entity refers to the process of preferentially isolating desired entities over undesired entities. In as far as this relates to isolation of the desired entities, the terms “isolating” and “sorting” are equivalent. The method of the present invention permits the sorting of desired genetic elements from pools (libraries or repertoires) of genetic elements which contain the desired genetic element. Selecting is used to refer to the process (including the sorting process) of isolating an entity according to a particular property thereof.

In a highly preferred application, the method of the present invention is useful for sorting libraries of genetic elements. The invention accordingly provides a method according to preceding aspects of the invention, wherein the genetic elements are isolated from a library of genetic elements encoding a repertoire of gene products. Herein, the terms “library”, “repertoire” and “pool” are used according to their ordinary signification in the art, such that a library of genetic elements encodes a repertoire of gene products. In general, libraries are constructed from pools of genetic elements and have properties which facilitate sorting.

Initial selection of a genetic element from a genetic element library using the present invention will in most cases require the screening of a large number of variant genetic elements. Libraries of genetic elements can be created in a variety of different ways, including the following.

Pools of naturally occurring genetic elements can be cloned from genomic DNA or cDNA (Sambrook et al., 1989); for example, phage antibody libraries, made by PCR amplification repertoires of antibody genes from immunised or unimmunised donors have proved very effective sources of functional antibody fragments (Winter et al., 1994; Hoogenboom, 1997). Libraries of genes can also be made by encoding all (see for example Smith, 1985; Parmley and Smith, 1988) or part of genes (see for example Lowman et al., 1991) or pools of genes (see for example Nissim et al., 1994) by a randomised or doped synthetic oligonucleotide. Libraries can also be made by introducing mutations into a genetic element or pool of genetic elements ‘randomly’ by a variety of techniques in vivo, including; using mutator strains of bacteria such as E. coli mutD5 (Liao et al., 1986; Yamagishi et al., 1990; Low et al., 1996); using the antibody hypermutation system of B-lymphocytes (Yelamos et al., 1995). Random mutations can also be introduced both in vivo and in vitro by chemical mutagens, and ionising or UV irradiation (see Friedberg et al., 1995), or incorporation of mutagenic base analogues (Freese, 1959; Zaccolo et al., 1996). Random mutations can also be introduced into genes in vitro during polymerisation for example by using error-prone polymerases (Leung et al., 1989).

Further diversification can be introduced by using homologous recombination either in vivo (see Kowalczykowski et al., 1994) or in vitro (Stemmer, 1994a; Stemmer, 1994b).

According to a further aspect of the present invention, therefore, there is provided a method of in vitro evolution comprising the steps of:
(a) selecting one or more genetic elements from a genetic element library according to the present invention;
(b) mutating the selected genetic element(s) in order to generate a further library of genetic elements encoding a repertoire to gene products; and
(c) iteratively repeating steps (a) and (b) in order to obtain a gene product with enhanced activity.

Mutations may be introduced into the genetic elements(s) as set forth above.

The genetic elements according to the invention advantageously encode enzymes, preferably of pharmacological or industrial interest, activators or inhibitors, especially of biological systems, such as cellular signal transduction mechanisms, antibodies and fragments thereof, and other binding agents (e.g. transcription factors) suitable for diagnostic and therapeutic applications. In a preferred aspect, therefore, the invention permits the identification and isolation of clinically or industrially useful products. In a further aspect of the invention, there is provided a product when isolated by the method of the invention.

The selection of suitable encapsulation conditions is desirable. Depending on the complexity and size of the library to be screened, it may be beneficial to set up the encapsulation procedure such that 1 or less than 1 genetic element is encapsulated per microcapsule. This will provide the greatest power of resolution. Where the library is larger and/or more complex, however, this may be impracticable; it may be preferable to encapsulate several genetic elements together and rely on repeated application of the method of the invention to achieve sorting of the desired activity. A combination of encapsulation procedures may be used to obtain the desired enrichment.

Theoretical studies indicate that the larger the number of genetic element variants created the more likely it is that a molecule will be created with the properties desired (see Perelson and Oster, 1979 for a description of how this applies to repertoires of antibodies). Recently it has also been confirmed practically that larger phage-antibody repertoires do indeed give rise to more antibodies with better binding affinities than smaller repertoires (Griffiths et al., 1994). To ensure that rare variants are generated and thus are capable of being selected, a large library size is desirable. Thus, the use of optimally small microcapsules is beneficial.

The largest repertoire created to date using methods that require an in vivo step (phage-display and LacI systems) has been a 1.6×10¹¹ clone phage-peptide library which required the fermentation of 15 litres of bacteria (Fisch et al., 1996). SELEX experiments are often carried out on very large numbers of variants (up to 10¹⁵).

Using the present invention, at a preferred microcapsule diameter of 2.6 μm, a repertoire size of at least 10¹¹ can be selected using 1 ml aqueous phase in a 20 ml emulsion.

In addition to the genetic elements described above, the microcapsules according to the invention will comprise further components required for the sorting process to take place. Other components of the system will for example comprise those necessary for transcription and/or translation of the genetic element. These are selected for the requirements of a specific system from the following; a suitable buffer, an in vitro transcription/replication system and/or an in vitro translation system containing all the necessary ingredients, enzymes and cofactors, RNA polymerase, nucleotides, nucleic acids (natural or synthetic), transfer RNAs, ribosomes and amino acids, and the substrates of the reaction of interest in order to allow selection of the modified gene product.

A suitable buffer will be one in which all of the desired components of the biological system are active and will therefore depend upon the requirements of each specific reaction system.

Buffers suitable for biological and/or chemical reactions are known in the art and recipes provided in various laboratory texts, such as Sambrook et al., 1989.

The in vitro translation system will usually comprise a cell extract, typically from bacteria (Zubay, 1973; Zubay, 1980; Lesley et al., 1991; Lesley, 1995), rabbit reticulocytes (Pelham and Jackson, 1976), or wheat germ (Anderson et al., 1983). Many suitable systems are commercially available (for example from Promega) including some which will allow coupled transcription/translation (all the bacterial systems and the reticulocyte and wheat germ TNT™ extract systems from Promega). The mixture of amino acids used may include synthetic amino acids if desired, to increase the possible number or variety of proteins produced in the library. This can be accomplished by charging tRNAs with artificial amino acids and using these tRNAs for the in vitro translation of the proteins to be selected (Ellman et al., 1991; Benner, 1994; Mendel et al., 1995).

After each round of selection the enrichment of the pool of genetic elements for those encoding the molecules of interest can be assayed by non-compartmentalised in vitro transcription/replication or coupled transcription-translation reactions. The selected pool is cloned into a suitable plasmid vector and RNA or recombinant protein is produced from the individual clones for further purification and assay.

In a preferred aspect, the internal environment of a microcapsule may be altered by addition of reagents to the oil phase of the emulsion. The reagents diffuse through the oil phase to the aqueous microcapsule environment. Preferably, the reagents are at least partly water-soluble, such that a proportion thereof is distributed from the oil phase to the aqueous microcapsule environment. Advantageously, the reagents are substantially insoluble in the oil phase. Reagents are preferably mixed into the oil phase by mechanical mixing, for example vortexing.

The reagents which may be added via the oil phase include substrates, buffering components, factors and the like. In particular, the internal pH of microcapsules may be altered in situ by adding acidic or basic components to the oil phase.

The invention moreover relates to a method for producing a gene product, once a genetic element encoding the gene product has been sorted by the method of the invention. Clearly, the genetic element itself may be directly expressed by conventional means to produce the gene product. However, alternative techniques may be employed, as will be apparent to those skilled in the art. For example, the genetic information incorporated in the gene product may be incorporated into a suitable expression vector, and expressed therefrom.

The invention also describes the use of conventional screening techniques to identify compounds which are capable of interacting with the gene products identified by the first aspect of the invention. In preferred embodiments, gene product encoding nucleic acid is incorporated into a vector, and introduced into suitable host cells to produce transformed cell lines that express the gene product. The resulting cell lines can then be produced for reproducible qualitative and/or quantitative analysis of the effect(s) of potential drugs affecting gene product function. Thus gene product expressing cells may be employed for the identification of compounds, particularly small molecular weight compounds, which modulate the function of gene product. Thus host cells expressing gene product are useful for drug screening and it is a further object of the present invention to provide a method for identifying compounds which modulate the activity of the gene product, said method comprising exposing cells containing heterologous DNA encoding gene product, wherein said cells produce functional gene product, to at least one compound or mixture of compounds or signal whose ability to modulate the activity of said gene product is sought to be determined, and thereafter monitoring said cells for changes caused by said modulation. Such an assay enables the identification of modulators, such as agonists, antagonists and allosteric modulators, of the gene product. As used herein, a compound or signal that modulates the activity of gene product refers to a compound that alters the activity of gene product in such a way that the activity of the gene product is different in the presence of the compound or signal (as compared to the absence of said compound or signal).

Cell-based screening assays can be designed by constructing cell lines in which the expression of a reporter protein, i.e. an easily assayable protein, such as β-galactosidase, chloramphenicol acetyltransferase (CAT), green fluorescent protein (GFP) or luciferase, is dependent on gene product. Such an assay enables the detection of compounds that directly modulate gene product function, such as compounds that antagonise gene product, or compounds that inhibit or potentiate other cellular functions required for the activity of gene product.

The present invention also provides a method to exogenously affect gene product dependent processes occurring in cells. Recombinant gene product producing host cells, e.g. mammalian cells, can be contacted with a test compound, and the modulating effect(s) thereof can then be evaluated by comparing the gene product-mediated response in the presence and absence of test compound, or relating the gene product-mediated response of test cells, or control cells (i.e., cells that do not express gene product), to the presence of the compound.

In a further aspect, the invention relates to a method for optimising a production process which involves at least one step which is facilitated by a polypeptide. For example, the step may be a catalytic step, which is facilitated by an enzyme. Thus, the invention provides a method for preparing a compound or compounds comprising the steps of:
(a) providing a synthesis protocol wherein at least one step is facilitated by a polypeptide;
(b) preparing genetic elements encoding variants of the polypeptide which facilitates this step;
(c) compartmentalising genetic elements into microcapsules;
(d) expressing the genetic elements to produce their respective gene products within the microcapsules, and linking the gene products to their respective genetic elements such that not more than one molecule of gene product is linked to each genetic element;
(e) sorting the genetic elements which produce polypeptide gene product(s) having the desired activity; and
(f) preparing the compound or compounds using the polypeptide gene product identified in (g) to facilitate the relevant step of the synthesis.

By means of the invention, enzymes involved in the preparation of a compound may be optimised by selection for optimal activity. The procedure involves the preparation of variants of the polypeptide to be screened, which equate to a library of polypeptides as refereed to herein. The variants may be prepared in the same manner as the libraries discussed elsewhere herein.

(B) Selection Procedures

The system can be configured to select for RNA, DNA or protein gene product molecules with catalytic, regulatory or other activities.

(i) Selection for Catalysis

When selection is for catalysis, the genetic element in each microcapsule may comprise the substrate of the reaction. If the genetic element encodes a gene product capable of acting as a catalyst, the gene product will catalyse the conversion of the substrate into the product. Therefore, at the end of the reaction the genetic element is physically linked to the product of the catalysed reaction.

It may also be desirable, in some cases, for the substrate not to be a component of the genetic element. In this case the substrate would contain an inactive “tag” that requires a further step to activate it such as photoactivation (e.g. of a “caged” biotin analogue, (Sundberg et al., 1995; Pirrung and Huang, 1996)). The catalyst to be selected then converts the substrate to product. The “tag” is then activated and the “tagged” substrate and/or product bound by a tag-binding molecule (e.g. avidin or streptavidin) complexed with the nucleic acid. The ratio of substrate to product attached to the nucleic acid via the “tag” will therefore reflect the ratio of the substrate and product in solution.

The assay may be configured to result in a change in optical properties of the microcapsules or the genetic element itself. This facilitates flow sorting. The optical properties of genetic elements with product attached and which encode gene products with the desired catalytic activity can be modified by, for example:
(1) the product-genetic element complex having characteristic optical properties not found in the substrate-genetic element complex, due to, for example;
   (a) the substrate and product having different optical properties (many fluorogenic enzyme substrates are available commercially (see for example Haugland, 1996) including substrates for glycosidases, phosphatases, peptidases and proteases (Craig et al., 1995; Huang et al., 1992; Brynes et al., 1982; Jones et al., 1997; Matayoshi et al., 1990; Wang et al., 1990)), or
   (b) the substrate and product having similar optical properties, but only the product, and not the substrate binds to, or reacts with, the genetic element;
(2) adding reagents which specifically bind to, or react with, the product and which thereby induce a change in the optical properties of the genetic elements allowing their sorting (these reagents can be added before or after breaking the microcapsules and pooling the genetic elements). The reagents;
   (a) bind specifically to, or react specifically with, the product, and not the substrate, if both substrate and product are attached to the genetic element, or
   (b) optionally bind both substrate and product if only the product, and not the substrate binds to, or reacts with, the genetic element.

The pooled genetic elements encoding catalytic molecules can then be enriched by selecting for the genetic elements with modified optical properties.

An alternative is to couple the nucleic acid to a product-specific antibody (or other product-specific molecule). In this scenario, the substrate (or one of the substrates) is present in each microcapsule unlinked to the genetic element, but has a molecular “tag” (for example biotin, DIG or DNP or a fluorescent group). When the catalyst to be selected converts the substrate to product, the product retains the “tag” and is then captured in the microcapsule by the product-specific antibody. In this way the genetic element only becomes associated with the “tag” when it encodes or produces an enzyme capable of converting substrate to product. When all reactions are stopped and the microcapsules are combined, the genetic elements encoding active enzymes will be “tagged” and may already have changed optical properties, for example, if the “tag” was a fluorescent group. Alternatively, a change in optical properties of “tagged” genes can be induced by adding a fluorescently labelled ligand which binds the “tag” (for example fluorescently-labelled avidin/streptavidin, an anti-“tag” antibody which is fluorescent, or a non-fluorescent anti-“tag” antibody which can be detected by a second fluorescently-labelled antibody).

Alternatively, selection may be performed indirectly by coupling a first reaction to subsequent reactions that takes place in the same microcapsule. There are two general ways in which this may be performed. In a first embodiment, the product of the first reaction is reacted with, or bound by, a molecule which does not react with the substrate of the first reaction. A second, coupled reaction will only proceed in the presence of the product of the first reaction. A genetic element encoding a gene product with a desired activity can then be purified by using the properties of the product of the second reaction to induce a change in the optical properties of the genetic element as above.

Alternatively, the product of the reaction being selected may be the substrate or cofactor for a second enzyme-catalysed reaction. The enzyme to catalyse the second reaction can either be translated in situ in the microcapsules or incorporated in the reaction mixture prior to microencapsulation. Only when the first reaction proceeds will the coupled enzyme generate a product which can be used to induce a change in the optical properties of the genetic element as above.

This concept of coupling can be elaborated to incorporate multiple enzymes, each using as a substrate the product of the previous reaction. This allows for selection of enzymes that will not react with an immobilised substrate. It can also be designed to give increased sensitivity by signal amplification if a product of one reaction is a catalyst or a cofactor for a second reaction or series of reactions leading to a selectable product (for example, see Johannsson and Bates, 1988; Johannsson, 1991). Furthermore an enzyme cascade system can be based on the production of an activator for an enzyme or the destruction of an enzyme inhibitor (see Mize et al., 1989). Coupling also has the advantage that a common selection system can be used for a whole group of enzymes which generate the same product and allows for the selection of complicated chemical transformations that cannot be performed in a single step.

Such a method of coupling thus enables the evolution of novel “metabolic pathways” in vitro in a stepwise fashion, selecting and improving first one step and then the next. The selection strategy is based on the final product of the pathway, so that all earlier steps can be evolved independently or sequentially without setting up a new selection system for each step of the reaction.

Expressed in an alternative manner, there is provided a method of isolating one or more genetic elements encoding a gene product having a desired catalytic activity, comprising the steps of:
(1) expressing genetic elements to give their respective gene products;
(2) allowing the gene products to catalyse conversion of a substrate to a product, which may or may not be directly selectable, in accordance with the desired activity;
(3) optionally coupling the first reaction to one or more subsequent reactions, each reaction being modulated by the product of the previous reactions, and leading to the creation of a final, selectable product;
(4) linking the selectable product of catalysis to the genetic elements by either:
   a) coupling a substrate to the genetic elements in such a way that the product remains associated with the genetic elements, or
   b) reacting or binding the selectable product to the genetic elements by way of a suitable molecular “tag” attached to the substrate which remains on the product, or
   c) coupling the selectable product (but not the substrate) to the genetic elements by means of a product-specific reaction or interaction with the product; and
(5) selecting the product of catalysis, together with the genetic element to which it is bound, either by means of its characteristic optical properties, or by adding reagents which specifically bind to, or react specifically with, the product and which thereby induce a change in the optical properties of the genetic elements wherein steps (1) to (4) each genetic element and respective gene product is contained within a microcapsule.
   (ii) Selecting for Enzyme Substrate Specificity/Selectivity

Genetic elements encoding enzymes with substrate specificity or selectivity can be specifically enriched by carrying out a positive selection for reaction with one substrate and a negative selection for reaction with another substrate. Such combined positive and negative selection pressure should be of great importance in isolating regio-selective and stereo-selective enzymes (for example, enzymes that can distinguish between two enantiomers of the same substrate). For example, two substrates (e.g. two different enantiomers) are each labelled with different tags (e.g. two different fluorophores) such that the tags become attached to the genetic element by the enzyme-catalysed reaction. If the two tags confer different optical properties on the genetic element the substrate specificity of the enzyme can be determined from the optical properties of the genetic element and those genetic elements encoding gene products with the wrong (or no) specificity rejected. Tags conferring no change in optical activity can also be used if tag-specific ligands with different optical properties are added (e.g. tag-specific antibodies labelled with different fluorophores).

(iii) Selection for Regulation

A similar system can be used to select for regulatory properties of enzymes.

In the case of selection for a regulator molecule which acts as an activator or inhibitor of a biochemical process, the components of the biochemical process can either be translated in situ in each microcapsule or can be incorporated in the reaction mixture prior to microencapsulation. If the genetic element being selected is to encode an activator, selection can be performed for the product of the regulated reaction, as described above in connection with catalysis. If an inhibitor is desired, selection can be for a chemical property specific to the substrate of the regulated reaction.

There is therefore provided a method of sorting one or more genetic elements coding for a gene product exhibiting a desired regulatory activity, comprising the steps of:
(1) expressing genetic elements to give their respective gene products;
(2) allowing the gene products to activate or inhibit a biochemical reaction, or sequence of coupled reactions, in accordance with the desired activity, in such a way as to allow the generation or survival of a selectable molecule;
(3) linking the selectable molecule to the genetic elements either by
   a) having the selectable molecule, or the substrate from which it derives, attached to the genetic elements, or
   b) reacting or binding the selectable product to the genetic elements, by way of a suitable molecular “tag” attached to the substrate which remains on the product, or
   c) coupling the product of catalysis (but not the substrate) to the genetic elements, by means of a product-specific reaction or interaction with the product;
(4) selecting the selectable product, together with the genetic element to which it is bound, either by means of its characteristic optical properties, or by adding reagents which specifically bind to, or react specifically with, the product and which thereby induce a change in the optical properties of the genetic elements wherein steps (1) to (3) each genetic element and respective gene product is contained within a microcapsule.
   (iv) Selection for Optical Properties of the Gene Product

It is possible to select for inherent optical properties of gene products if, in the microcapsules, the gene product binds back to the genetic element, for example through a common element of the gene product which binds to a ligand which is part of the genetic element. After pooling the genetic elements they can then be sorted using the optical properties of the bound gene products. This embodiment can be used, for example, to select variants of green fluorescent protein (GFP) (Cormack et al., 1996; Delagrave et al., 1995; Ehrig et al., 1995), with improved fluorescence and/or novel absoption and emmission spectra.

(v) Flow Sorting of Genetic Elements

In a preferred embodiment of the invention the genetic elements will be sorted by flow cytometry. A variety of optical properties can be used to trigger sorting, including light scattering (Kerker, 1983) and fluorescence polarisation (Rolland et al., 1985). In a highly preferred embodiment the difference in optical properties of the genetic elements will be a difference in fluorescence and the genetic elements will be sorted using a fluorescence activated cell sorter (Norman, 1980; Mackenzie and Pinder, 1986), or similar device. In an especially preferred embodiment the genetic element comprises of a nonfluorescent nonmagnetic (e.g. polystyrene) or paramagnetic microbead (see Formusek and Vetvicka, 1986), optimally 0.6 to 1.0 μm diameter, to which are attached both the gene and the groups involved in generating a fluorescent signal:
(1) commercially available fluorescence activated cell sorting equipment from established manufacturers (e.g. Becton-Dickinson, Coulter) allows the sorting of up to 10⁸ genetic elements (events) per hour;
(2) the fluorescence signal from each bead corresponds tightly to the number of fluorescent molecules attached to the bead. At present as little as few hundred fluorescent molecules per particle can be quantitatively detected;
(3) the wide dynamic range of the fluorescence detectors (typically 4 log units) allows easy setting of the stringency of the sorting procedure, thus allowing the recovery of the optimal number of genetic elements from the starting pool (the gates can be set to separate beads with small differences in fluorescence or to only separate out beads with large differences in fluorescence, dependant on the selection being performed;
(4) commercially available fluorescence-activated cell sorting equipment can perform simultaneous excitation at up to two different wavelengths and detect fluorescence at up to four different wavelengths (Shapiro, 1983) allowing positive and negative selections to be performed simultaneously by monitoring the labelling of the genetic element with two (or more) different fluorescent markers, for example, if two alternative substrates for an enzyme (e.g. two different enantiomers) are labelled with different fluorescent tags the genetic element can labelled with different fluorophores dependent on the substrate used and only genes encoding enzymes with enantioselectivity selected.
(5) highly uniform derivatised and non-derivatised nonmagnetic and paramagnetic microparticles (beads) are commercially available from many sources (e.g. Sigma, and Molecular Probes) (Formusek and Vetvicka, 1986).
   (vi) Multi-Step Procedure

It will be also be appreciated that according to the present invention, it is not necessary for all the processes of transcription/replication and/or translation, and selection to proceed in one single step, with all reactions taking place in one microcapsule. The selection procedure may comprise two or more steps. First, transcription/replication and/or translation of each genetic element of a genetic element library may take place in a first microcapsule. Each gene product is then linked to the genetic element which encoded it (which resides in the same microcapsule), for example via a gene product-specific ligand such as an antibody. The microcapsules are then broken, and the genetic elements attached to their respective gene products optionally purified. Alternatively, genetic elements can be attached to their respective gene products using methods which do not rely on encapsulation. For example phage display (Smith, G. P., 1985), polysome display (Mattheakkis et al., 1994), RNA-peptide fusion (Roberts and Szostak, 1997) or lac repressor peptide fusion (Cull, et al., 1992).

In the second step of the procedure, each purified genetic element attached to its gene product is put into a second microcapsule containing components of the reaction to be selected. This reaction is then initiated. After completion of the reactions, the microcapsules are again broken and the modified genetic elements are selected. In the case of complicated multistep reactions in which many individual components and reaction steps are involved, one or more intervening steps may be performed between the initial step of creation and linking of gene product to genetic element, and the final step of generating the selectable change in the genetic element.

If necessary, release of the gene product from the genetic element within a secondary microcapsule can be achieved in a variety of ways, including by specific competition by a low-molecular weight product for the binding site or cleavage of a linker region joining the binding domain of the gene product from the catalytic domain either enzymatically (using specific proteases) or autocatalytically (using an integrin domain).

(vii) Selection by Activation of Reporter Gene Expression IN Situ

The system can be configured such that the desired binding, catalytic or regulatory activity encoded by a genetic element leads, directly or indirectly to the activation of expression of a “reporter gene” that is present in all microcapsules. Only gene products with the desired activity activate expression of the reporter gene. The activity resulting from reporter gene expression allows the selection of the genetic element (or of the compartment containing it) by any of the methods described herein.

For example, activation of the reporter gene may be the result of a binding activity of the gene product in a manner analogous to the “two hybrid system” (Fields and Song, 1989). Activation can also result from the product of a reaction catalysed by a desirable gene product. For example, the reaction product can be a transcriptional inducer of the reporter gene. For example arabinose may be used to induce transcription from the araBAD promoter. The activity of the desirable gene product can also result in the modification of a transcription factor, resulting in expression of the reporter gene. For example, if the desired gene product is a kinase or phosphatase the phosphorylation or dephosphorylation of a transcription factor may lead to activation of reporter gene expression.

(viii) Amplification

According to a further aspect of the present invention the method comprises the further step of amplifying the genetic elements. Selective amplification may be used as a means to enrich for genetic elements encoding the desired gene product.

In all the above configurations, genetic material comprised in the genetic elements may be amplified and the process repeated in iterative steps. Amplification may be by the polymerase chain reaction (Saiki et al., 1988) or by using one of a variety of other gene amplification techniques including; Qb replicase amplification (Cahill, Foster and Mahan, 1991; Chetverin and Spirin, 1995; Katanaev, Kumasov and Spirin, 1995); the ligase chain reaction (LCR) (Landegren et al., 1988; Barany, 1991); the self-sustained sequence replication system (Fahy, Kwoh and Gingeras, 1991) and strand displacement amplification (Walker et al., 1992).

Various aspects and embodiments of the present invention are illustrated in the following examples. It will be appreciated that modification of detail may be made without departing from the scope of the invention.

All documents mentioned in the text are incorporated by reference.

### EXAMPLES

We describe herein a novel IVC strategy based on ‘microbead-display’ in which repertoires of microbeads are created, each displaying a gene and the protein it encodes (FIG. 1A). These beads can be selected on the basis of the binding activity of the displayed polypeptide (Sepp, A., Tawfik, D. S. and Griffiths, A. D., manuscript submitted), or, as described here, selected for catalysis with a soluble substrate under multiple turnover conditions and in a chosen reaction environment (FIG. 1B).

Here we demonstrate the utility of this novel approach by selecting genes that encode the bacterial phosphotriesterase (PTE) from Pseudomonas diminuta and Flavobacterium sp. This enzyme catalyses the hydrolysis of a range of organophosphate trimesters but its best substrate in terms of both k_{cat}, and K_{M} is paraoxon (FIG. 3A) (for review see [Raushel, 2000]). PTE is a remarkably efficient enzyme: although thought to have evolved within 50 years or so, its catalytic performances are remarkable, k_{cat}, for paraoxon hydrolysis is 2280 s⁻¹, and the k_{cat}/K_{M} of 6.2×10⁷ M⁻¹ s⁻¹ is close to the diffusion-controlled limit [Hong, 1999].

First, we illustrate the fundamentals of the strategy via a model selection. The PTE gene was spiked into a large excess of ΔOPD genes that encode a catalytically inactive protein (FIG. 2) and then enriched for using the strategy described in FIG. 1. Next, we select libraries derived from the PTE gene. Despite PTE being a very proficient enzyme, directed evolution using this newly described system yielded a mutant with much improved turnover number.

Results

Model Selections

Microbead-Display—Linking Genes to the Proteins they Encode.

6×10⁸ 1 μm diameter streptavidin-coated beads coated with biotinylated anti-HA antibodies were incubated with biotinylated OPD genes encoding the enzyme phosphotriesterase (PTE)[Mulbry, 1989], or with biotinylated ΔOPD genes which encode an inactive protein (FIG. 2), or mixtures of these genes, at a ratio of 0.3 genes per bead. These microbeads were resuspended in a cell-free translation mixture, compartmentalised in a water-in-oil emulsion, and incubated for 4 hours at 23° C. to allow translation of the genes and capture of the protein by the anti-HA antibody. As the translated proteins encoded by both genes (OPD and ΔOPD) are tagged at their C-terminus with the HA epitope (FIG. 2), microbeads isolated from these emulsions display the proteins encoded by the genes attached to them (FIG. 1A). After the emulsion was broken, the beads were resuspended in a buffer suitable for the enzymatic reaction and which contained zinc and carbonate ions to allow the captured inactive apo-enzyme to assemble into the catalytically active PTE metaloenzyme [Hong, 1995]. The metaloenzyme immobilised on these beads can hydrolyse paraoxon (FIG. 3A) and hence is active (Table 1, 3b). On average, about 30 PTE molecules were captured per bead, corresponding to more than half of the total in vitro expressed protein. When genes encoding proteins with no PTE activity (e.g., ΔOPD) were immobilised on beads and translated, no paraoxon hydrolysis was observed (Table 1, 3a). Translation and capture of the enzyme onto the beads proceeds in bulk solution and in the aqueous compartments of the emulsion with comparable efficiency (see Table 1, 3c vs 3f). However, compartmentalisation in an emulsion ensures that translated proteins become attached to the genes that encode them (via the bead) and not to other genes (attached to other beads). This physical linkage between the gene and the encoded protein can be used to select proteins or peptides for binding (Sepp, A., Tawfik, D. S. and Griffiths, A. D., manuscript submitted), as with other display approaches such as mRNA-peptide fusion and phage- or ribosome-display [Pluckthun, 2000; Sidhu, 2000; Griffiths, 1998 #31; Georgiou, 1997 #28; Wittrup, 2001; Schatz, 1996 #69; Amstutz, 2001; Keefe, 2001]. In contrast to all other display technologies, however, compartmentalisation also enables the selection of display libraries directly for enzymatic activity as described below.

The Compartmentalised Selection of Microbeads for Enzymatic Activity

To select for enzymatic activity, the microbead-displayed gene-protein complexes created in the first emulsion (FIG. 1A) were re-compartmentalised in a second emulsion as described in FIG. 1B. The caged-biotinylated substrate EtNP-cgB (FIG. 3B) was then added to the oil phase from where it diffuses into the aqueous droplets. This substrate is a close derivative of paraoxon (FIG. 3A) where one of the ethyl groups is replaced by a linker connected to caged-biotin. Indeed, the hydrolysis of EtNP-cgB is catalysed by PTE with kinetic parameters that are comparable to those of paraoxon (K_{M}=17 μM, k_{cat}=160 s⁻¹)for one of the two enantiomers of the chiral phosphotriester, whilst the other is hydrolysed ˜4000 fold slower). Caging prevents the biotinylated substrate from binding to the avidin-coated beads thus allowing it to interact with the bead-displayed enzyme in a soluble form. The emulsions were incubated for 16 hours to allow the hydrolysis of the substrate to be completed in those droplets containing the PTE enzyme (FIG. 3B). The emulsion was then irradiated to yield the biotinylated product or substrate that binds to the avidin-coated bead in the compartment (FIG. 3B). Consequently, beads carrying the PTE gene and the active enzyme become labelled with the product whilst beads carrying the ΔOPD gene, and hence a catalytically inactive protein, are labelled with an intact substrate. This way of coupling of genotype to phenotype—namely, of genes encoding an enzyme becoming labelled with the product of its activity—is the basis of selection for catalysis using IVC [Tawfik, 1998 #80]. Such linkage cannot be obtained in bulk solution where the product can diffuse freely and thus becomes attached, via the microbead, to any gene, not necessarily to the gene that encoded it.

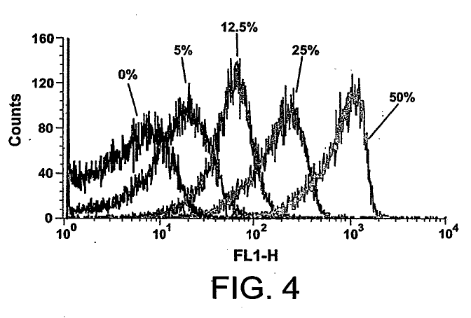

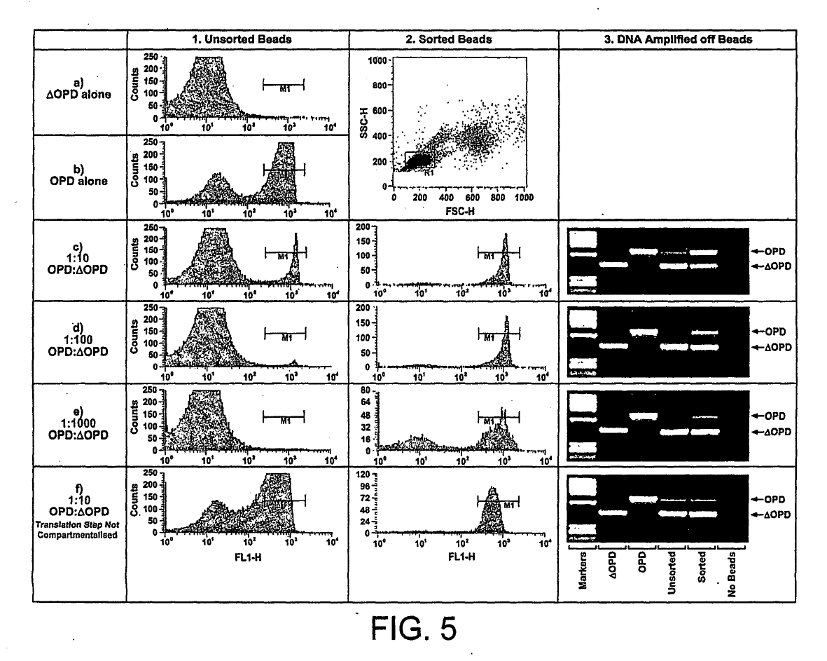

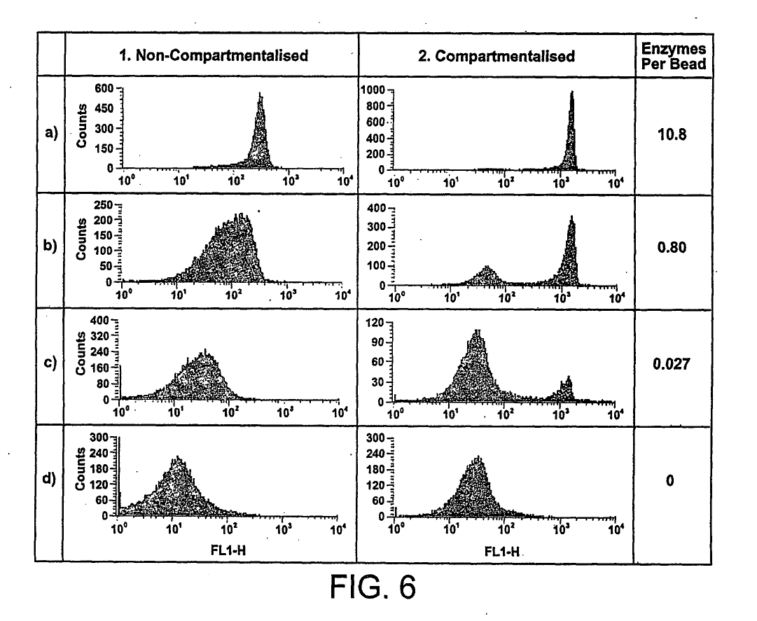

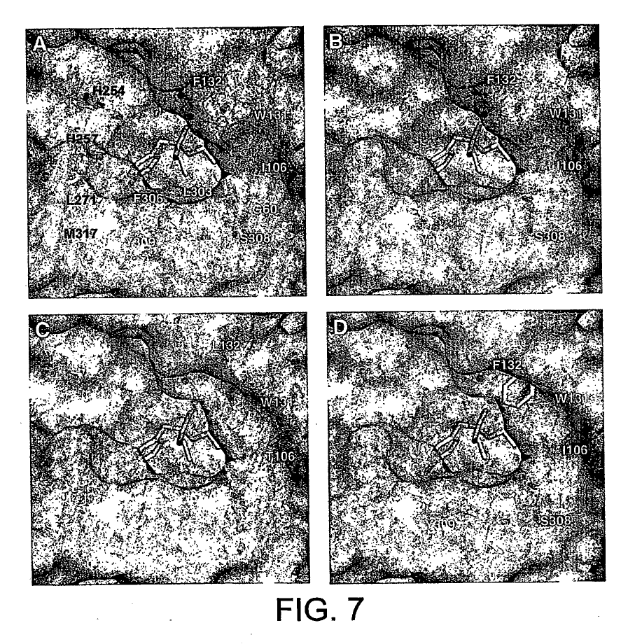

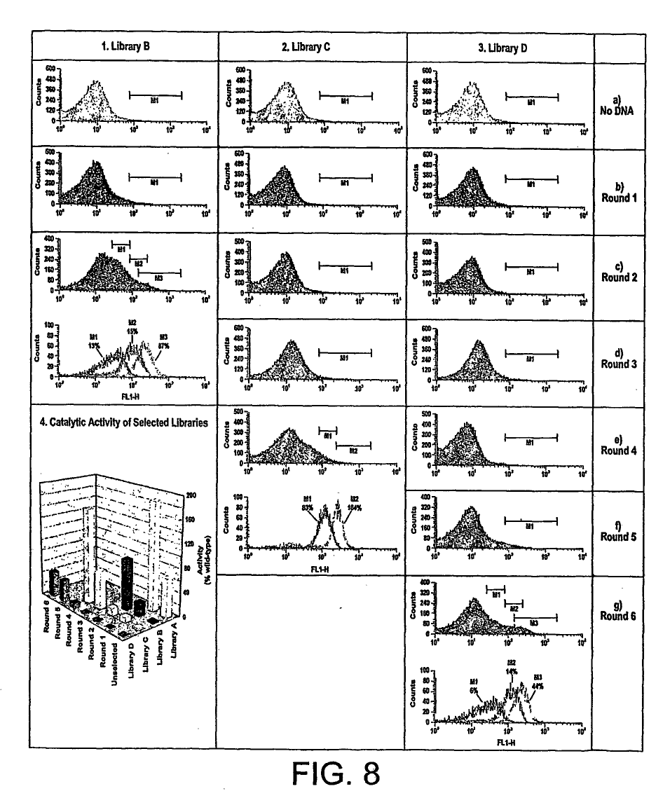

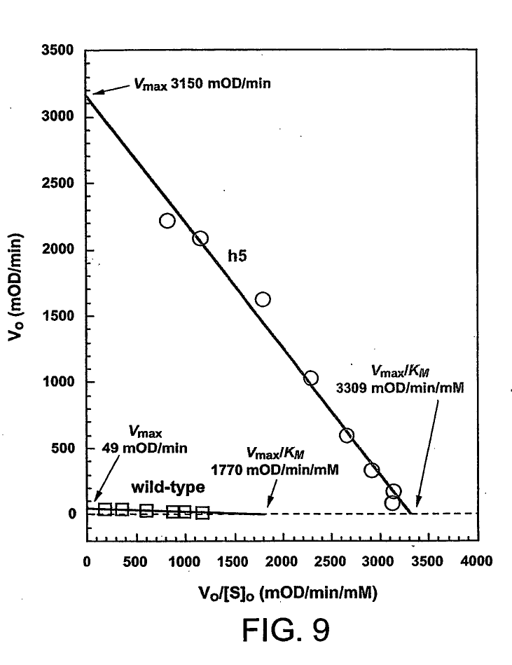

Once the coupling of product and substrate to the microbeads was completed, the emulsions were broken and the beads were fluorescently labelled using anti-product antibodies—namely, antibodies that bind the phosphodiester product (Et-B) but not the unhydrolysed substrate (EtNP-B) (FIG. 3B). The beads can then be analysed and sorted by flow cytometry as the mean fluorescence of beads coated with the 50% phosphodiester product (Et-B) (corresponding to the hydrolysis of only one of the two enantiomers of the chiral phosphotriester) is 129-fold higher than the mean fluorescence of beads coated with the substrate (EtNP-B) alone (FIG. 4). Indeed, flow cytometry can also distinguish between beads with different substrate:product ratios (FIG. 4). Hence, relatively small differences in the amount of product on beads can be translated into relatively large enrichments by sorting using suitable gates.

Flow cytometry revealed that beads carrying the ΔOPD gene (encoding an inactive protein), carry the substrate and, as expected, exhibit low fluorescence (FIG. 5, 1a). In contrast, beads carrying the OPD genes, onto which, following translation and capture, the active PTE enzyme is attached, carry the product and thus are highly fluorescent (FIG. 5, 1b). Not all the beads are highly fluorescent as, on average, only one in three beads had a gene attached. When the OPD gene was spiked into an excess of ΔOPD genes, a mixture of ‘positive’ (high fluorescence) and ‘negative’ (low fluorescence) beads was observed (FIG. 5, 1c-e). The percentage of positive beads (Table 1, 4) correlates very well with the fraction of the OPD gene in the starting mixture of genes (Table 1, 1). Given however, that at most one third of the beads should carry a gene, the number of positive beads is higher than expected. For example, the emulsion prepared with OPD genes should only have yielded 33% rather than 74.5% of positives (Table 1, 4; FIG. 5, 1b). This is almost certainly due to some compartments, in either the first or second emulsion, containing more than one bead.

To demonstrate the enrichment of genes encoding the PTE enzyme, 10⁵ single, unagregated, high fluorescence beads (gated through R1 and M1) from the experiments with 1:10, 1:100 or 1:1000 starting ratios of OPD:ΔOPD genes (FIG. 5, 1c-e) were sorted by flow cytometry. Analysis of the sorted beads by flow cytometry showed that enrichment had been efficient (Table 1, 5; FIG. 5, 2). The genes attached to the sorted and unsorted beads were amplified by PCR using primers (FIG. 2A) that anneal to both the OPD and ΔOPD genes (Table 1, 6; FIG. 5, 3). The ratio of genes before and after selection indicated up to 200 fold enrichment of the OPD gene following selection (Table 1, 7). These results not only demonstrate that compartmentalisation in the second emulsion can be used to select for enzymatic activity, but also that genes were linked, via microbeads, to the proteins they encode in the first emulsion. Indeed, when translation was performed in bulk solution rather then in an emulsion, the translated enzymes distributed between the beads regardless if they carried the OPD or ΔOPD genes. Consequently, most of the beads ended up with at least one enzyme molecule bound, became labelled with product and were selected as positives (FIG. 5, 1f). However, when the DNA on the selected beads (FIG. 5, 2f) was amplified, no enrichment for the OPD gene was observed (FIG. 5, 3f; Table 1, f).

Compartmentalisation Allows Single Enzyme Molecules to be Identified

The beads in the selection experiments described above carry ˜30 PTE enzyme molecules each (Table 1). But in fact, owing to the small volume of its compartments, the system allows a bead displaying only a single PTE molecule to be identified and sorted. To demonstrate the above, beads carrying different numbers of PTE molecules were subjected to the enzymatic selection procedure described above (FIG. 1B), either emulsified or in bulk solution (FIG. 6).

When coated with 10.8 PTE molecules/bead, all beads appeared to be highly fluorescent or ‘positive’. When less PTE molecules were coated and reactions performed in bulk solution, the enzyme concentration was too low to catalyse the complete conversion of substrate into product. The fluorescence was accordingly reduced, and, due to the equal distribution of substrate and product over the entire population of beads, a single population of beads was observed (FIG. 6, 1b-1c). However, when the very same beads were compartmentalised, two populations were observed: ‘positive’ beads that were as fluorescent as the ones obtained with 10.8 PTEs per bead, and ‘negative’ beads similar to the ones observed when no PTE is coated on the beads (FIG. 62b-2c). This demonstrates the effects of compartmentalisation. When there are far less PTE molecules than beads—for example, 1 PTE per 37 beads (FIG. 6, 2c), the vast majority of beads carry no PTE molecules and exhibit low fluorescence, and the rest carry only one PTE molecule, and are highly fluorescent. Thus, owing to the very small volume of these compartments (˜5 femtolitre), a single PTE molecule is present at high enough a concentration (˜0.2 nM) to allow the complete conversion of substrate into product, and a bead carrying a single PTE molecule can easily be identified and sorted.

Library Selections

Construction of Phosphotriesterase Libraries

Three substrate binding pockets (designated the large, small and leaving group pockets) within the active site of PTE have been assigned following the determination of PTE's structure[Vanhooke, 1996](FIG. 7A). The small subsite is thought to be defined primarily by the side chains of Gly-60, Ile-106, Leu-303, and Ser-308 and the large subsite consists mainly of His-254, His-257, Leu-271, and Met-317. The leaving group subsite is thought to be surrounded by Trp-131, Phe-132, Phe-306 and Tyr-309 and forms the entrance to the active site. We created four gene libraries by randomising codons in the wild-type OPD gene (which encodes PTE the enzyme). The codons randomised were Ile-106, Trp-131, Phe-132, Ser-308 and Tyr-309 (FIG. 7B). These residues define the entrance to the active site and the small subsite. Phe-306 and Leu-303 were not randomised as they are also involved in forming the large subsite and Gly-60 was left unchanged so as not to further reduce the size of the small subsite. The libraries were: Library A, Ile-106 randomised (diversity 32); Library B, Ile-106, Ser-308 and Tyr-309 randomised (diversity 3.3×10⁴); Library C, Ile-106, Trp-131, and Phe-132 randomised (diversity 3.3×10⁴); Library D, Ile-106, Trp-131, Phe-132, Ser-308 and Tyr-309 randomised (diversity 3.4×10⁷).

Selection of Phosphotriesterase Libraries

For selection of each library, 2×10⁸ molecules of linear DNA, with a triple biotin at each end (and made by PCR), were attached to 6×10⁸ streptavidin-coated beads (i.e. at 0.33 genes per bead) and selected for the ability to hydrolyse the phosphotriester substrate EtNP-cgB (FIG. 3B) as outlined in FIG. 1 and the model selections described above. The use of a triple biotin at each end of the DNA did not inhibit expression and provided a very stable link to the beads and resistance against exonucleases present in the in vitro translation extract (data not shown). Indeed, although beads were lost during selection (on average ˜12% of the beads were recovered after the two emulsifications), the recovery of DNA per bead was 57% based on quantitative PCR.

In the first round of selection, sorting was performed with a gate (M1) set to include no more than 1% of false positives (as determined by flow cytometry of beads that were not coated with DNA; FIG. 8, row a). 10⁵ high fluorescence beads were collected from Libraries A, B and C, and 5×10⁵ beads from Library D. This corresponded to sorting of a total of ˜5×10⁷ beads from Library D. In subsequent rounds of selection 10⁵ beads were collected for all libraries.

After each round of selection the DNA was amplified off the sorted beads by nested PCR. To prevent the accumulation of PCR artifacts that can arise after multiple rounds of amplification, the amplified DNA was digested with NcoI and SacI to yield the OPD gene (FIG. 2) and ligated into the expression vector to re-append the T7 promoter, ribosome binding site and terminator. The genes for the next round of selection were amplified directly from the ligated plasmid (without cloning or transformation) with the original (triply biotinylated) primers that prime the vector in regions outside the annealing sites of the primers used for nested PCR.

The libraries were taken through between one and six rounds of selection. Enrichment for genes encoding active phosphotriesterases was followed by flow cytometry of beads.

In the first round of selection of Library A, which is only randomised at a single codon, about one third of the beads formed a low fluorescence population but the rest were part of a higher fluorescence population (data not shown) indicating the presence of a large percentage of active sequences in the unselected library. This library was not selected further. For Libraries B and C (each with 3 codons randomised) a significant population of high fluorescence beads became visible by the second and fourth rounds of selection respectively (FIG. 8, 1c and 2e). For the Library D (with the highest diversity of 3.4×10⁷), a significant population of high fluorescence beads became visible by the sixth round of selection (FIG. 8, 3g).

In the final round of selection, several different gates were used to sort positive beads from each library. These gates (M1, M2 and M3) are shown in FIG. 8, 1c, 2e and 3g together with the flow cytometric analysis of the beads sorted through each gate.

In addition, the DNA from each round of selection was translated in vitro, incubated with Zn²⁺ to assemble the PTE metalloenzyme, and phosphotriesterase activity measured using 0.25 mM paraoxon (FIG. 8, panel 4). In parallel, the wild-type OPD gene and the unselected libraries DNA were also translated at the same DNA concentration. The activity of unselected Library A was already 66% of wild-type, increasing to 186% of wild-type after one round of selection. The activity of unselected Libraries B and C were 2.2% and 2.5% of wild-type, respectively. The activity rose steadily through successive rounds of selection with Library B reaching 87% of wild-type activity after round 2 and Library C reaching 164% of wild-type activity after round 4. Library D, the most diverse, had barely detectable activity (0.14% of wild-type) before selection but this rose to 44% of wild-type activity after round 6. The fact that the activities of two of the libraries (A and C) rose to above wild-type suggested that they may contain clones that were more active than wild-type under these assay conditions. The phosphotriesterase activity observed after translation of pools of sorted genes from the final round of selection varied with the gate used to sort the beads (FIG. 8, 1c, 2e and 3g), higher fluorescence beads yielding higher phosphotriesterase activity.

The unselected libraries and the libraries after the final round of selection (FIG. 8), were cloned into the pIVEX vector and transformed into E. coli. DNA from individual colonies was amplified by PCR. This DNA, and the wild-type OPD gene were translated in vitro and phosphotriesterase activity measured with paraoxon as substrate. Before selection 60% of the Library A clones analysed had detectable activity (≧0.1 mOD/min/μl IVT) with a mean activity 24% of wild-type. Libraries B, C, and D also contained significant numbers of clones with detectable activity (15%, 45% and 6% respectively), but the mean activity of these positive clones was much lower than wild-type (1.45%, 0.21% and 0.18% respectively). After the final round of selection the percentage of positive clones was 83% (Library 1), 26% (Library B), 23% (Library C) and 14% (Library D). Table 2 shows the activities and sequences of 35 clones taken at random from these selected libraries (and found to be pure clones by analysis of the sequencing chromatograms). The mean activity of these selected clones relative to wild-type were 72% (Library A), 13% (Library B), 74% (Library C) and 31% (Library D). These mean activities are in general agreement with the activities found in the pooled selected libraries (FIG. 8, panel 4). Hence, although the increase in the percentage of clones with detectable activity was modest there was a clear preferential enrichment for genes giving rise to higher phosphotriesterase activity in all the libraries. Indeed, several clones (b5, d5 c4 and h5) were significantly more active than wild-type. The percentage of positive clones found after sorting beads through different gates in the final round of selection (FIG. 8, 1c, 2e and 3g) did not vary significantly, nor was there a significant difference in the activities of the individual clones. This despite a clear difference in the activities from the pooled genes selected through different gates (FIG. 8, 1c, 2e and 3g). We suspect that the differences between the activity observed upon translation of the pools, vs. the average of individual genes isolated from these pools, are due to the relatively small sample sizes of individual clones analysed and the large effect a few highly active clones (e.g., h5) can have on the activity of the pool of genes.

Sequencing of active clones from the final round of selection showed that none had the wild-type sequence (Table 2).

Kinetic Characterisation of Selected Mutants Reveals a Phosphotriesterase with a very High k_{cat}.

The kinetic parameters for ten of the PTE mutants described in Table II were determined using paraoxon as substrate (Table III). The majority had a k_{cat} higher than wild-type PTE and for one of these (h5) k_{cat} was 2.8×10⁵ s⁻¹, which is 123-fold higher than wild-type PTE.

The K_{M} for all the mutants was increased (from 5- to 143-fold relative to wild-type) and only the two mutants with the fastest k_{cat}(h5 and b5), had a k_{cat}/K_{M} higher than wild-type. Data obtained with EtNP-cgB (FIG. 3B) also indicated that both k_{cat} and K_{M} were increased in clones with higher than wild-type activity (e.g., h5). However, the limited solubility and availability of EtNP-cgB prevented a full kinetic analysis.

In Vitro Selections for Enzymes

In Nature, repeated rounds of mutation, recombination and selection have generated enzymes and other proteins with remarkable properties. Darwinian evolution can also be applied in vitro to reproduce and study natural evolution and generate novel proteins with tailor-made properties [Soumillion, 2001; Petrounia, 2000; Georgiou, 2000; Ness, 2000; Pluckthun, 2000; Wahler, 2001; Griffiths, 2000]. Both processes require a link between genotype (a nucleic acid that can be replicated) and phenotype (a functional trait such as binding or catalytic activity) [Griffiths, 2000]. In vitro, this linkage is usually achieved by physically linking genes to the proteins they encode by a variety of techniques, including display on phage, viruses, bacteria and yeast, plasmid-display, ribosome-display and mRNA-peptide fusion. These ‘display technologies’, have proven highly successful in the generation of binding proteins [Pluckthun, 2000; Sidhu, 2000; Griffiths, 1998 #31; Georgiou, 1997 #28; Wittrup, 2001; Schatz, 1996 #69; Amstutz, 2001; Keefe, 2001].

In contrast to selections for binding, selection of enzymes by display approaches has met with little success. Indirect selections—by binding to transition state analogues or enzyme inhibitors—have generally failed to produce potent catalysts [Griffiths, 2000]. Single-turnover, intramolecular selections of enzymes displayed on phage were demonstrated but these impose severe limitations [Griffiths, 2000; Atwell, 1999]. To evolve proficient enzymes, the selection (or screen) should be simultaneous and direct for all enzymatic properties: substrate recognition, formation of a specific product, rate acceleration and turnover (the ability of a single active-site to catalyse the conversion of numerous substrate molecules). The only efficient catalytic, multiple-turnover selection so far described involved selection of OmpT variants using a positively charged fluorogenic substrate which binds to the negatively charged surface of E. coli allowing them to be sorted by flow cytometry [Olsen, 2000]. Although OmpT is a normal E. coli outer membrane protein this technique could potentially be extended to other heterologous enzymes displayed on the surface of E. coli [Georgiou, 1997 #28].

Direct selection for all enzymatic properties can be achieved by compartmentalisation in cells (as in nature). Directed evolution experiments can be performed by, for example, cloning and expressing a gene library in bacteria and screening 10³-10⁵ clones in a plate assay using a fluorogenic or chromogenic substrate. However, crossing long evolutionary distances, and in particular evolving completely novel proteins and activities, requires much larger libraries [Griffiths, 2000; Keefe, 2001]. In these cases, selection (namely a parallel screen where only genes encoding proteins with desired activity survive) rather than screening of discrete clones or genes is clearly the method of choice. Unfortunately, in vivo selections are usually (but not always[Firestine, 2000]) restricted to functions that affect the viability of the organism and are often complicated by the complex intracellular environment and the need to clone and transform the gene-library. In addition, very large libraries (>10¹² genes) are easily handled only in vitro. There is little doubt therefore, that purely in vitro systems will eventually prove advantageous [Fastrez, 1997 #23; Pluckthun, 2000; Minshull, 1999 #53].

While several completely in vitro selection systems are available for the selection of proteins for binding [Pluckthun, 2000; Roberts, 1999 #65], IVC is currently the only way of selecting directly for enzymatic activity. To date, however, IVC has only been applied for the selection of enzymes for which DNA is the substrate (and the gene and the substrate reside on the same molecule), both the enzymatic reaction and translation take place in the same environment, and where selection was not necessarily for multiple turnover (since the enzyme was in molar excess relative to the substrate) [Tawfik, 1998 #80].

Selecting Enzymes by In Vitro Compartmentalisation

Here we describe a much more general mode of selection with IVC allowing the selection of enzymes that catalyse the conversion of soluble, non-DNA substrates under multiple turnover conditions and in a reaction environment of choice. It is based on creating libraries of proteins displayed on microbeads by translation in an emulsion (FIG. 1A). Like any other display-library, libraries created by IVC can be selected for ligand-binding: either by displaying the polypeptides and the genes encoding them on microbeads as described here (FIG. 1A) (Sepp, A., Tawfik, D. S. and Griffiths, A. D., manuscript submitted) or by translation of biotinylated genes encoding peptides fused to streptavidin [Doi, 1999 #22].

By re-compartmentalising the microbead-display libraries in a second emulsion they can be selected for catalysis, as described here (FIG. 1B). Selection for enzymatic activity is completely detached from translation and can take place in any buffer or at any temperature and is not complicated by the complex milieu of a cell or an in vitro translation system. For example, the phosphotriesterase selected here is translated as an inactive apo-protein and is assembled later in the course of the enzymatic selection. Even thermophilic enzymes could potentially be evolved since emulsions similar to the ones used here are stable at 99° C. [Ghadessy, 2001].

Selection is also performed on a soluble substrate (at essentially any given concentration) and is for turnover. A comparison of the fluorescence of the ‘positive’ beads selected here, produced by ˜30 (FIG. 5), or even a single (FIG. 6) enzyme molecule per bead, with the fluorescence of beads coated with known ratios of substrate and product (FIG. 4), indicated that almost all the active enantiomer of the substrate had been converted to product.

In the selections, >10⁶ substrate molecules were added per bead, each enzyme, therefore, must have catalysed the formation of ˜10⁶ product molecules. At the same time, the system is sensitive enough to detect partial conversion of substrate into product (≧5%; FIG. 4), and, typically not one but rather >30 enzyme molecules are displayed on each bead. Thus, the system presented here has the potential to select enzymes that are at least 300-fold less active than wild-type PTE. Based on PTE having k_{cat}, for EtNP-cgB of 160 s⁻¹, and assuming the rate of base-catalysed hydrolysis of EtNP-cgB to be 2.4×10⁻⁷ s⁻¹ at pH 8.5, as for paraoxon[Dumas, 1989]), this represents a dynamic range (in terms of k_{cat}/k_{uncat}) of at least 2×10⁶ up to ˜10⁹ that is probably sufficient to improve or alter the activity of of almost most existing enzymes [Griffiths, 2000; Fastrez, 1997 #23].

Here we used a substrate that was modified by coupling to caged-biotin, but a further advantage of compartmentalisation is that it should allow an unmodified substrate to be used, provided that the selected reaction is coupled to a second reaction that uses a caged substrate. In addition, by using substrates modified with a photo-labelling group such as 2,3,5,6-tetrafluoro-4-azizobenzoic acid (ATFB) [Keana, 1990] the enzymatic selection strategy could potentially be used with other types of display-libraries, for example libraries displayed on phage or ribosomes. For example, the modified PTE substrate EtNP-ATFB (FIG. 3C) with (ATFB; 2,3,5,6-tetrafluoro-4-azizobenzoic acid) upon irradiation becomes attached to any protein or DNA present in the compartments and has been used to label microbeads as an alternative to the caged biotinylated substrate EtNP-cgB (data not shown).

Flow cytometry has previously been used to select libraries of enzymes displayed on the surface of bacteria [Olsen, 2000] and can also be used to select microbead-display libraries, as demonstrated here. It can dramatically increase screening throughput since modern instruments can handle up to 100,000 events per second (http://www.cytomation.com), but also has other potential advantages [Georgiou, 2000].

Flow cytometry does impose an upper limit of ˜10⁹ on the size of libraries that can be selected. However, larger libraries could be selected by affinity purification of product coated beads (for example using paramagnetic beads coated with anti-product antibodies).

PTE Library Selections

We have demonstrated the utility of this technique by selecting improved enzymes from libraries based on the bacterial enzyme phosphotriesterase (PTE) that catalyses the hydrolysis of a wide range of organophosphorus pesticides and nerve agents [Dumas, 1989].

X-ray crystallographic studies of PTE in complex with substrate analogues reveals the binding pocket to be predominantly hydrophobic [Vanhooke, 1996; Benning, 2000]. Three sub-sites has beeen described: the so called large and small pockets and the leaving group pocket that defines the entrance to the active site (FIG. 7A). We created a PTE libraries with five codons randomised (overall diversity of 3.4×10⁷). The residues randomised were Ile-106, Trp-131, Phe-132, Ser-308 and Tyr-309 (FIG. 7B). These residues form the entrance to the active site (the leaving group site) and the small subsite. Selection of all four libraries resulted in an enrichment for phosphotriesterase activity as seen by the gradual appearance of significant numbers of high fluorescence (product coated) beads, and the increased phosphotriesterase activity following the translation of the pool of selected genes with each round of selection (FIG. 8). In the case of the largest library (D) there was little detectable phosphotriesterase activity before selection. Nevertheless, 6% of clones tested had low, but detectable, phosphotriesterase activity (on average 0.18% of wild-type activity). Although the percentage of genes in the library with detectable activity had risen only slightly by the sixth and final round of selection (to 14%), the mean activity had risen by more than 150 fold (to 31% of wild-type activity). A similar pattern was seen with the smaller libraries but less rounds of selection were required due to the higher percentage of genes with significant phosphotriesterase activity in the unselected libraries. Hence, there was a clear enrichment for clones with higher phosphotriesterase activity at the expense of those with lower activity.

The Newly-Evolved PTE Clones

When single clones with phosphotriesterase activity were analysed after the final round of selection none had the wild-type sequence (Table 2). Indeed, at position 106, the wild-type residue, isoleucine did not appear to be favoured and was present in only two clones that both exhibit very low activity. Instead, the commonest residues at position 106 were the serine and threonine. At position 132, the wild-type residue, phenylalanine, was the second most common residue, but the most common was leucine. At the three other positions mutated, although many different amino acids were seen, the wild-type residue seemed to be favoured and was the most common. For example, the most common residue selected at position 131 was tryptophan (9/21), as in wild-type PTE, and this residue prevailed in the more active clones. Although the substrate binding site of PTE is predominantly hydrophobic, a potential hydrogen bond between N^{ε1} of Trp-131 and the phosphoryl oxygen has been identified from crystallographic studies of PTE complexed with substrate analogs [Vanhooke, 1996; Benning, 2000]. At position 308, a range of different residues were observed, but the most common was the wild-type residue, serine (7/23). Similarly, at position 309, many different residues were observed, but tyrosine, the wild-type residues, was the most common (5/23).

The above sequence preference was reflected in the kinetic properties of the selected phosphotriesterases (Table 3). The clone (h5) with the fastest turnover number was isolated from Library D in which five codons were randomised. However, this clone only had two mutations relative to wild-type PTE, Ile-106 to Thr and Phe-132 to Leu. Of the clones analysed kinetically, the majority had a k_{cat} higher than wild-type PTE and a lower k_{cat}/K_{M}. Only two clones (h5 & b5) exhibited a k_{cat}/K_{M} higher than wild-type. The increase in K_{M} observed in all the selected mutants (5-143 fold) suggests that selection occurred under substrate concentrations that are significantly higher than the wild-type's K_{M}[Fersht, 1999]. However, the caged-biotinylated substrate EtNP-cgB was added to a maximum concentration of 50 μM (assuming that all the substrate had partitioned into the aqueous droplets), a concentration that is very similar to the K_{M} of both paraoxon and EtNP-cgB. It is therefore possible that the effective substrate concentration in the aqueous compartments is higher than expected, perhaps due to surface effects at the water-oil interface in the emulsion.

The Extremely Fast PTE Mutant

The clone (h5) with the fastest turnover is quite remarkable. Wild-type PTE is already a very efficient enzyme: k_{cat} for paraoxon hydrolysis (FIG. 3A) is 2280 s⁻¹, and the k_{cat}/K_{M} of 6.2×10⁷ M-1 s⁻¹ is close to the diffusion-controlled limit [Hong, 1999]. Despite this, PTE clone h5 has k_{cat} of 2.8×10⁵ S⁻¹, 123-fold faster than wild-type PTE and a k_{cat}/K_{M} of three times higher than wild-type (3.4×10⁸M⁻¹s⁻¹). In the most efficient enzymes, the k_{cat}/K_{M} can be as high as 3×10⁸ M⁻s⁻¹, in which case the rate-determining step for k_{cat}/K_{M} is thought to be the diffusion-controlled encounter of the enzyme and the substrate[Fersht, 1999]. Thus, PTE clone h5 is one of the most efficient enzymes ever described. There are some enzymes with a faster k_{cat}, (notably catalase; k_{cat} 4×10⁷ s⁻¹, [Ogura, 1955]), but to the best of our knowledge, the fastest hydrolase previously described is acetylcholinesterase (k_{cat}, 1.4×10⁴ s⁻¹ and k_{cat}/K_{M} 1.6×10⁸ M⁻¹ s⁻¹; [Rosenberry, 1975]) and PTE clone h5 has a k_{cat}, 20 times faster than this.

The origins of PTE-h5's remarkable k_{cat} are currently under investigation. The high k_{cat}/K_{M} implies that the transition state is bound as strongly as in the wild-type. The rate-limiting step for hydrolysis of paraoxon by wild-type PTE is thought to be related to product dissociation rather than bond breaking [Caldwell, 1991]. Hence, the increased rate could be due to faster product release and the higher K_{M} may reflect a decreased affinity not only for the substrate but also for the product. The Phe-132 to Leu mutation found in PTE h5 may facilitate the release of product by opening up the entrance to the active site (FIG. 7). In wild type PTE, Trp-131 and Phe-132 stack on top of each other. The side chain of Leu is smaller than that of Phe and cannot stack against TRp-131. It may allow many more degrees of rotational freedom for the underlying Trp-131 and thereby facilitate the exit of product from the active site. However, the full explanation is probably not quite so simple. In a previous study by Raushel and colleagues, each residue in the substrate binding site of PTE was mutated individually to Ala, and Ile-106, Phe-132 and Ser-308 were also mutated to Gly [Chen-Goodspeed, 2001; Chen-Goodspeed, 2001]. However, no large improvements in the rate of paraoxon hydrolysis were observed with any of these mutations. Most mutations had only a small effect on maximum velocity (Vₘₐₓ) The largest increase in Vₘₐₓ seen with a single mutation (Ile-106 to Gly) was 4-fold and the largest increase with a double (or triple) mutation to Ala or Gly was 5.5-fold (Ile-106 and Ser-308 to Gly). Simultaneous mutations of Ile-106 and Phe-132, either both to Ala or both to Gly, gave only a 3-fold increase in Vₘₐₓ. Hence, the precise nature of the substitutions at each of these positions is of great importance.

It is quite difficult, even with the benefit of hindsight, to rationalise exactly why the two mutations in PTE clone h5 lead to such a highly efficient enzyme. However, this highlights the benefits of using a strategy based on high throughput screening or selection to create enzymes with improved activities. The libraries used in this study were designed using structural information from crystallographic studies but selection from a wide repertoire allowed a large margin of error in the design strategy.

Materials and Methods

Synthesis of Genes

The OPD gene encoding the phosphotriesterase (PTE) enzyme was amplified from Flavobacterium sp. (strain ATCC 27551)[Mulbry, 1989] by PCR using primers OPD-Flag-Bc and OPD-HA-Fo and cloned into NcoI and SacI cut pIVEX2.2b Nde (Roche) to give pIVEX-OPD (FIG. 2) which expresses PTE with N-terminal Flag[Chiang, 1993 #91] and C-terminal HA[Field, 1988] epitope tags. pIVEX-OPD was digested with HincII and NotI, treated with Klenow polymerase, and religated creating pIVEX-ΔOPD, in which the OPD gene has a 258 base-pair (bp) in-frame deletion.

The linear, biotinylated DNAs for selection (FIG. 2A) were prepared by PCR amplification of the above vectors. Two 600 μl, PCR reactions (using Super Taq; HT Biotechnology) were performed using primers pIV-B1 and LMB2-1-tribiotin (FIG. 2A; Table 2) and ˜0.1 μg of either pIVEX-OPD or pIVEX-ΔOPD as template. The reactions were cycled 30 times (94° C., 0.5 min, 50° C., 0.5 min, 72° C., 2.0 min) with a final step at 72° C. for 7 min. Each amplified DNA was purified using Wizard PCR Preps (Promega), analysed by agarose gel electrophoresis and quantified by measuring the absorbance at 260 nm.

Synthesis of PTE Libraries

Four libraries were created by saturation mutagenesis with either one, three or five codons replaced with NNS (where N is an equimolar mixture of A, T, G & C and S is a mixture of G & C). The libraries were created by a series of PCR reactions. Library A (which has Ile-106 randomised) was created by re-assembling the OPD gene from two fragments. The ′N-terminal fragment was prepared by PCR amplification of the OPD gene with primers LMB2-1-biotin and LibA-Fo which anneals to the OPD gene upstream of Ile-106 to append the diversified codon (NNS) replacing Ile-106 and appending a BsmBI site. The ′C-terminal fragment was prepared by PCR amplification of the OPD gene with primers pIV-B1 and LibA-Bc which anneals downstream of Ile106 and also appends a BsmBI site. The two fragments were digested with BsmBI and gel-purified. 10¹² molecules each of the ‘N-terminal’ and ‘C-terminal’ fragments were mixed, ligated overnight using T4 DNA ligase and captured on 2 mg Streptavidin M-280 Dynabeads (Dynal). The supernatant (containing the unligated ‘C-terminal’ fragment) was removed and the beads (containing the ligated OPD gene and unligated ′N-terminal fragment) rinsed. The ligation efficiency, determined using a P³²-labelled ′C-terminal fragment was 10-20%, thus yielding >10⁹ full-length OPD genes per ligation.

Library B (which has Ile-106, Ser-308 and Tyr-309 randomised) was created by PCR amplification of Library A with primers LMB2-9-biotin and LibB-Fo which anneals to the OPD gene upstream of Ser-308, contains two NNS codons replacing Ser-308 and Tyr-309, and appends a BsmBI site, to give the ′N-terminal fragment. The ‘C-terminal’ fragment was prepared by PCR amplification of the OPD gene with primers pIVB-1 and LibB-Bc which anneals downstream of Tyr-309 and appends a BsmBI site. These fragments were digested and ligated as above.

Library C (which has Ile-106, Trp-131 and Phe-132) was created as above by ligating an ′N-terminal fragment (created with primers LMB2-8-biotin and LibC-Fo which anneals to the OPD gene upstream of Trp-131, contains two NNS codons replacing Trp-131 and Phe-132) and a ′C-terminal fragment (generated with primers pIVB-1 and LibC-Bc which anneals downstream of Phe-132).

Library D (which has Ile-106, Trp-131, Phe-132, Ser-308 and Tyr-309 randomised), was created by PCR amplifying the ligation of Library B (which has Ile-106, Ser-308 and Tyr-309 randomised) and Library C (Ile-106, Trp-131 and Phe-132 randomised) and (see above) with primers pIV-B5 and LMB2-5-biotin. The amplified DNA was gel purified and digested with BcII (which cuts between Phe-132 and Ser-308). The 'digested fragments were gel purified and ligated as above.

The ligated OPD genes from all four libraries were PCR amplified with primers LMB2-9 and pIV-B9, digested with NcoI and SacI and 10¹¹ molecules ligated into 5×10¹⁰ molecules pIVEX2.2b Nde (Roche) cut with the same enzymes. The ligation reactions (each containing >10⁹ molecules of ligated vector) were PCR amplified with primers pIV-B1-tribiotin and LMB2-1-tribiotin. The 200 μl PCR reactions were cycled 30 times (94° C., 0.5 min, 50° C., 0.5 min, 72° C., 2.0 min) with a final step at 72° C. for 7 min and the full-length genes (1829 base pairs) gel-purified. Sequencing of this library DNA as is, and of DNA amplified from individual clones after transformation of the ligations into E. coli TG1[Gibson, 1984] (at least five from each library) confirmed that sequence diversity had been incorporated into the libraries as expected.

Synthesis and Characterisation of PTE Substrates

Caged-biotin was synthesised by following published procedures [Pirrung, 1996 #61; Sundberg, 1995] and then coupled via a linker to the p-nitrophenyl-ethyl phosphotriester substrate to give the caged biotinylated substrate EtNP-cgB (FIG. 3B). The biotinylated substrate EtNP-B (FIG. 3B) was created using biotin-N-hydroxysuccinimide ester to couple biotin to the p-nitrophenyl-ethyl phosphotriester substrate via a linker. Detailed procedures will be published elsewhere. Both substrates were hydrolysed in the presence of recombinant Zn-assembled PTE[Dumas, 1989] to release p-nitrophenol and the corresponding products (Et-cgB and Et-B, respectively). The substrate used in the selections (EtNP-cgB) has a K_{M} similar, or even lower than paraoxon (17 μM) and a k_{cat}, of 160s⁻¹ that is ˜13 fold lower than paraoxon[Dumas, 1989]. With both substrates, only 50% of the substrate was hydrolysed by PTE. The remaining half of the substrate could be hydrolysed by either base or PTE, albeit, at a rate which is ˜4000 times slower than the first half. This is due to these substrates being comprised of two enantiomers of the chiral phosphotriester. Indeed, PTE is known to exhibit enantioselectivity by preferring Sₚ over Rₚ enantiomers of various phosphotriesters by 1-130 fold[Hong, 1999].

Generation of Anti-Product Antibodies

Following a previously published procedure[Tawfik, 1993] a p-nitrophenyl-ethyl phosphotriester substrate with a suitable linker was coupled to KLH (keyhole limpet hemocyanin) and BSA (bovine serum albumin) and the p-nitrophenyl ester and subsequently hydrolysed to give the phosphodiester product. Antibodies were elicited in rabbits by immunisation with EtBG-KLH (hapten density=14) using published protocols[Tawfik, 1993 #79; Tawfik, 1997] in the laboratory of Prof. Z Eshhar (Weizmann Institute of Science, Rehovot, Israel). Sera were tested by ELISA for binding to both the substrate conjugate EtNPBG-BSA (Hd=8.5) and the corresponding product conjugate (EtBG-BSA; Hd=8.5). The first bleed from one of the immunised rabbits, when diluted 500 fold or more, exhibited the desired selectivity: it gave a high signal (by ELISA) when incubated with the product conjugate and a low background (<20%) with the substrate conjugate. Diluting the sera in COVAp buffer (2M NaCl, 0.04% Tween-20, 10 mM phosphate, 0.1 mM p-nitrophenol, pH˜6.5) gave even higher selectivity with the background levels on the substrate conjugate (EtNPBG-BSA) dropping below 5%.

Selections for PTE

Generation of Microbead-Display Libraries in the First Emulsion

Coating of beads with anti-HA antibodies and DNA. The biotin-binding capacity of the beads used in this procedure needs to be sufficiently high to accommodate the anti-tag antibodies, the gene, and the substrate or product of the selected reaction. Here we used 0.95 μm streptavidin-coated polystyrene beads with a capacity of 0.545 μg biotin-FITC/mg beads (Bangs, #CP01N, ˜2×10⁷ beads/μl; Lot 4771). All bead manipulations were performed in 1.7 ml MaxyClear tubes (Axygen). 195 μl of these beads were spun down in a microfuge (3 minutes, 6.5 krpm), rinsed twice (by resuspension and centrifugation) in 200 μl PBS/T/Hp (50 mM Sodium Phosphate pH 7.5, 100 mM NaCl, 0.1% Tween 20, 8 mg/ml Heparin, sodium salt) and resuspended in the same buffer. After sonication for 1 minute (in a Branson 200 Ultrasonic Cleaner), 46 μl of 50 μg/ml biotinylated anti-HA antibody (Roche, biotinylated 3F10) were added (to give 2500 antibody molecules per bead) and the beads incubated for 2 hours at 20° C., mixing at 1400 rpm for 10 seconds every minute using a Thermomixer comfort (Eppendorf). (The PTE gene applied here was also tagged with the Flag epitope at its amino terminus (FIG. 2). However, capture by anti-N-Flag M5 antibodies was less efficient, yielding on average ˜1 PTE molecule per bead). The beads were split into six aliquots of ˜6×10⁸ beads each. The linear biotinylated DNAs (see above) were diluted to 0.66 nM in 100 μg/ml λ-Hind-III markers (New England Biolabs). In addition, 0.66 nM solutions containing both ‘OPD genes’ and ‘ΔOPD genes’ were created by mixing the above solution at the ratios indicated in Table 1. 0.5 μl of 0.66 nM DNA were added to each bead aliquot at a ratio of 0.33 genes/bead. The beads were incubated 16 hours at 7° C., mixing at 1400 rpm for 10 seconds every minute. The beads were rinsed, once in 100 μl PBS/T/Hp, once in 100 μl 5 mM Tris-Acetate pH 8.0, 1 mg/ml Heparin (sodium salt), resuspended in 18 μl of 5 mM Tris-Acetate pH 8.0 and sonicated for 1 minute.

In vitro translation (IVT) and emulsification. A fresh Span/Triton oil mix was prepared (0.5% w/w Triton-X100 (Fischer) and 4.5% w/w Span 80 (Fluka) in light mineral oil (Sigma)). Each 18 μl of coated beads (˜6×10⁸ beads) were mixed with 2 μl 5 mM Methionine and 35 μl EcoPro T7 reaction mix (from the EcoPro T7 in vitro translation system; Novagen) on ice. Samples were added to 0.5 ml of oil mix while stirring at 1600 rpm. Stirring was continued for 5 minutes on ice. All reactions were incubated for 4 hours at 23° C. The emulsions were subsequently transferred to microfuge tubes and spun for 7 minutes at 20800 g. The oil phase was removed leaving the white pellet (the concentrated unbroken emulsion). 1 ml of mineral oil was added and the emulsion resuspend. The tube was re-spun and the oil phase removed. The oil rinse was repeated once more to break the emulsion and the oil and aqueous phase removed. The beads were resuspended in 200 μl PBS/T (50 mM Sodium Phosphate pH 7.5, 100 mM NaCl, 0.1% Tween 20) and 1 ml of mineral oil was added. The mixture was vortexed and spun down (3 minutes at 9000 g). The oil and aqueous phase were removed, the bead pellet resuspended in 200 μl PBS/T and extracted three times with 1 ml hexane. Residual hexane was removed by spinning 5 minutes at room temperature in a Speedvac (Savant) After spinning 3 minutes at 9000 g the supernatant was removed and the beads rinsed: 3 times with 100 μl PBS/T plus 5 mM EDTA and 5 mM EGTA (the second rinse was incubated for 10 minutes); once with 100 μl PBS/T/Hp; and once with 100 μl Tris/Carb/Zn buffer (50 mM Tris-HCl, 10 mM Potassium Carbonate, 25 μM ZnCl₂, pH 8.5).

The beads were resuspended in 60 μL of Tris/Carb/Zn buffer, sonicated for 1 minute and put on ice for the selection for PTE activity (see below).

Assaying phosphotriesterase activity on the beads. A sample from the above suspension (˜4×10⁷ beads) was used to assay the PTE activity of the in vitro translated enzyme captured on these beads. The bead suspension was assembled by incubation in Tris/CO₂/Zn buffer (50 mM Tris-HCl, 25 μM ZnCl₂, 10 mM K₂CO₃, pH 8.5) for 16 hours at 4° C. Activity of the assembled enzyme was measured with 0.25 mM paraoxon in 50 mM Tris-HCl pH 8.5 by monitoring the release of the p-nitrophenolate product at 405 nm[Dumas, 1989]

Selection for Phosphotriesterase Activity in the Second Emulsion

Re-emulsification and uncaging. The bead display libraries, prepared as above, were added to 0.5 ml of ice-cold Span/Triton oil mix while stirring at 1150 rpm. Stirring was continued for 3 minutes on ice, and the emulsion was then homogenised for 3 minutes at 11 krpm using an Ultra-Turrax T8 Homogeniser (IKA) with a 5 mm diameter dispersing tool. The resulting emulsion was incubated at 25° C. for 10 minutes. A methanolic solution of the caged-biotinylated substrate EtNP-cgB (1.75 mM) was added to give a concentration of 5 μM in the oil and the emulsions mixed briefly. All samples were then incubated at 25° C. for 16 hours to complete the PTE-catalysed hydrolysis of the substrate. To uncage the substrate and product within the water droplets of these emulsions, 0.5 ml of 7.5 mM acetic acid in Span/Triton oil were and the emulsions mixed. The emulsions were transferred to a 24-well plate (Nunc) and irradiated 4 minutes on ice with gentle stirring (200 rpm) using a B100 AP 354 nm UV lamp (UVP) from ˜5 cm distance, The emulsions were then incubated for 30 minutes at 25° C. and broken as described above. After removing residual hexane beads were rinsed 3 times with PBS/T by resuspension and centrifugation. Finally the beads were resuspended in 100 μl PBS/T and sonicated for 1 minute.

Labelling beads with anti-product antibodies and flow cytometry The anti-product rabbit serum (see above) was diluted 1:30 in COVAp buffer plus 1.5 mg/ml BSA. 100 μl of diluted serum were added to each bead suspension (˜2×10⁸ beads) and incubated for 1.5 hrs. The beads were rinsed twice with PBS/T by centrifugation (in a microfuge, 2 minutes at 14 krpm) and resuspended in 100 μl of PBS/T. 100 μl of 50 ng/μl FITC-labelled goat anti rabbit Fab (Min. X; Jackson) in COVAp buffer plus 1.5 mg/ml BSA were added to each bead suspension and incubated for 1 hr. The beads were rinsed twice with PBS/T, resuspended in 100 μl of PBS/T and sonicated for 2 minutes. as above. The beads were diluted by adding 1.4 ml PBS/T (to give ˜10⁸ beads/ml) and run on a MoFlo flow cytometer (Cytomation) at ˜20,000 events per second, with a 100 μm nozzle tip, exciting with a 488 nm Argon Ion laser (Coherent Innova 70; 10 Watts max. CW Output) at full power, and measuring emission passing a 530±20 nm bandpass filter. Single, unagregated beads were gated using forward- and side-scatter and ˜100,000 high fluorescence ‘positive’ beads were collected. In addition, 50 μl of unselected beads were further diluted into 1 ml PBS (to give ˜5×10⁶ beads/ml), and 50 μl of selected beads were diluted into 250 μl PBS/T. 100,000 events from the unselected samples and 10,000 events from the selected samples were analysed by flow cytometry using a FACScan cytometer (BD) to check enrichment for high fluorescence ‘positive’ beads.

PCR amplification of the selected genes. ˜10⁵ beads from before and after sorting were transferred to 1.7 ml microfuge tubes and spun 5 minutes at 14 krpm. All but ˜20 μl supernatant were removed and 200 μl PCR buffer added. The procedure was repeated twice and the beads finally resuspended and sonicated in a total volume of 100 μl of PCR buffer to give ˜10³ beads/μl. 50 μl PCR reactions were performed using Super Taq (HT Biotechnology), primers OPDPCRB5 and OPDPCRF5 (FIG. 2A; Table 4) and 25 μl bead suspensions from above. The reactions were cycled 22 times (94° C., 0.5 min, 50° C., 0.5 min, 72° C., 2.0 min) with a final step at 72° C. for 7 min. The amplification was repeated with 50 μl PCR reactions using nesting primers OPDPCRB6 and OPDPCRF6 (FIG. 2A; Table 2) and 1 μl of the first PCR reaction as template. These reactions were cycled 33 times (94° C., 0.5 min, 50° C., 0.5 min, 72° C., 2.0 min) with a final step at 72° C. for 7 min. 1.25 μl of amplified DNA was analysed by electrophoresis on a 2.5% agarose gel containing ethidium bromide with 500 μg φX174 cut HaeIII DNA markers.

Selection of PTE Libraries

2×10⁸ genes amplified with primers pIV-B1-tribiotin and LMB2-1-tribiotin from Libraries A, B, C and D were each coated onto ˜6×10⁸ streptavidin-coated polystyrene beads (Bangs, #CP01N, ˜2×10⁷ beads/μl; Lot 5016; binding capacity 0.64 μg biotin-FITC/mg beads), and selected using the same protocol as for the model selection above. In the first round of selection flow sorting was used to collect 100,000 high fluorescence beads from Libraries A, B and C and 500,000 beads from Library D using a gate set to include only ˜1% of beads which were not coated with DNA. Genes were amplified off selected beads by PCR as for the model selections but using Pfu Turbo enzyme (Stratagene) and primers pIVB-8 and LMB-2-8 for the first PCR and pIVB-9 and LMB-2-9 for the subsequent nested PCR. The reactions were cycled 22 times for the first PCR and 33 times for the subsequent nested PCR (95° C., 0.5 min, 50° C., 0.5 min, 72° C., 2.0 min) with a final step at 72° C. for 10 min.

The PCRs were purified directly with Wizard PCR Preps (Promega), digested with NcoI and SacI, and 10¹¹ molecules ligated into 10¹⁰ molecules pIVEX2.2b Nde (Roche) cut with the same enzymes (as described for the preparation of the libraries above). 10⁹ molecules of vector from each of the ligations of Libraries A, B, C and D were PCR amplified (using Pfu Turbo polymerase) with primers pIV-B 1-tribiotin and LMB2-1-tribiotin in a 200 μl PCR reaction cycled 30 times (95° C., 0.5 min, 50° C., 0.5 min, 72° C., 2.0 min) with a final step at 72° C. for 10 min. The full-length genes (1829 base pairs) were gel purified as above.

Up to six rounds of selection were performed in total by repeating the above procedure except that after round one, only 100,000 high fluorescence beads were collected from sorting of each library. In addition, in the final round of selection several different gates were used to sort positive beads (see FIG. 8).

Assaying phosphotriesterase activity in the selected libraries. The wild-type OPD gene and DNA amplified from the ligations of the unselected libraries and the libraries after each round of selection were diluted to 4 nM in 25 μg/ml λ-Hind-III markers (New England Biolabs) and translated at a final concentration of 1 nM for 4 hours at 23° C. in a 10 μl in vitro translation reaction (EcoPro T7 in vitro translation system; Novagen). The Zn²⁺ metalloenzyme was assembled assembled by adding 30 μl Tris/CO₂/Zn buffer and incubating for 1.5 hours at room temperature. Activity of the assembled enzyme was measured with 0.25 mM paraoxon in 50 mM Tris-HCl pH 8.5 by monitoring the release of the p-nitrophenolate product at 405 nm[Dumas, 1989].

Characterisation of Selected Clones

1 or 2 μl of the ligations of the unselected and selected libraries were transformed into XL10-Gold Ultracompetent cells (Stratagene) and plated on TYE, 100 μg/ml ampicillin, 1% glucose plates. 384 individual colonies were picked from each ligation into a 384-well Large Volume Plate (Genetix) containing 2×TY, 100 μg/ml ampicillin, 8% glycerol, 1% glucose (75 μl per well) using a colony picking robot (Kaybee Systems), incubated overnight at 37° C. and stored at −70° C.

96 pin disposable replicators (15 mm, thin; Genetix) were used to transfer bacteria from the above 384-well plates into 50 μl PCR reactions using Super Taq polymerase (HT Biotechnology) and primers pIV-B1 and LMB2-1 set up in 96-well Thermo-Fast, Low Profile PCR Plates (Abgene). The plates were sealed with Adhesive PCR Film (Abgene) and (with heated lid on) incubated 94° C. for 10 min then cycling 30 times (94° C., 0.5 min, 50° C., 0.5 min, 72° C., 2.0 min) with a final step at 72° C. for 7 min. The average concentration of DNA, was determined by comparing to markers of known concentration on an agarose gel as above. The DNA from each well was diluted to ˜10 nM in 25 μg/ml ˜-Hind-III markers and translated at a final concentration of ˜2 nM for 6 hours at 30° C. in a 2.5 μl in vitro translation reaction (Rapid Translation System RTS 100, E. coli HY Kit; Roche Diagnostics) set up in a Thermo-Fast, 384-well PCR Plates (Abgene). The Zn²⁺ metalloenzyme was assembled by adding 15 μl Tris/CO₂/Zn buffer and incubating for 1.5 hours at room temperature. Activity of the assembled enzyme was measured with 0.25 mM paraoxon in 50 mM Tris-HCl pH 8.5 by monitoring the release of the p-nitrophenolate product at 405 nm[Dumas, 1989].

Clones showing detectable paraoxon hydrolysing activity (>0.1 mOD/min/μIVT) were re-amplified by PCR from the bacterial stocks as above. The DNA was purified using a QIAquick 96 PCR Purification Kit (Qiagen) into water and the DNA concentration determined by comparing to markers of known concentration on an agarose gel as above.

The DNA was sequenced using using primers T7 and pIV-B9 and also translated in vitro and assayed for paraoxon hydrolysis as above.

Kinetic Analysis of Selected Clones

PCR amplified DNA from the selected PTE clones was translated at 1 nM using the EcoPro T7 in vitro translation system and assembled as described above. Rates were measured in 50 mM Tris-HCl pH 8.5, with 0.02-4 μl of IVT and 0.014-3.6 mM paraoxon. K_{M} and vₘₐₓ were determined by fitting the data to the Michaelis-Menten model (Vₒ=vₘₐₓ[S]ₒ/([S]o+K_{M})) using KaleidaGraph. Assuming a k_{cat} of 2280s⁻¹ (Hong and Raushel, 1999), the vₘₐₓ found for wild-type PTE (0.4 μM/sec/μl IVT) corresponds to an enzyme concentration of 35 nM in the in vitro translation mix. The relative concentrations of the wild-type and mutant PTEs were determined by a sandwich ELISA based on the PTE possessing an N-terminal Flag tag and C-terminal HA tag (FIG. 2) and used to convert vₘₐₓ to k_{cat}(Table III). Microtitre plates (Nunc, Maxisorb) were coated with the anti-FLAG M5 antibody (Sigma; 3.5 μg/ml; overnight at 4° C.) and blocked with BSA. The IVT reactions were serially diluted (25 up to 225 fold) in PBS/T and incubated in the coated plates for 1 hour. The plates were rinsed and biotinylated anti-HA antibody (3F10; Roche; 0.5 μg/ml in PBS) was added, followed (after rinsing) by streptavidin-peroxidase (Sigma; diluted 4000 fold in PBS). The assay was developed using TNB (Nolge). A calibration curve made with in vitro translated wild-type PTE was used to determine the concentration of the mutants. Expression levels varied from 10% (h 11) to 480% (b5) relative to wild-type. Errors were +20% or lower, and generally higher than errors assigned to from the fit to the Michaelis-Menten model.

Tables

TABLE I
Creation of microbead-display libraries and selections for catalysis

| | | | | % of | % of | | |
|---|---|---|---|---|---|---|---|
| | | | | positive | positive | | |
| | | | | events in | events in | | |
| | | | | unsorted | sorted | Final ratio of | |
| | Starting | | Captured | beads^{d} | beads^{e} | OPD/ΔOPD | |
| | ratio of | | PTE | (FIG. 5, | (FIG. 5, | gene^{f} | Enrichment |
| | OPD/ΔOPD | Generation of | molecules | Column 1: | Column 2: | (FIG. 5, | for the |
| | genes^{a} | display libraries^{b} | per gene^{c} | region M1) | region M1) | Column 3) | OPD gene^{g} |
| 1 | ΔOPD alone | Compartmentalised | 0 | 0 | n.d. | n.d. | — |
| 2 | OPD alone | ″ | 31.5 | 74.3 | n.d | n.d | — |
| 3 | 1:10 | ″ | 3.6 | 11.4 | 93.6 | 1:0.7 | 14 |
| 4 | 1:100 | ″ | 0 | 1.08 | 86.6 | 1:2.1 | 47 |
| 5 | 1:1000 | ″ | 0 | 0.09 | 57.5 | 1:4.6 | 217 |
| 6 | 1:10 | Non-compartmentalised | 3.8 | 61.9 | 93.2 | 1:7.6 | 1.3 |

^{a}Mixtures of the OPD and ΔOPD genes (FIG. 2A) at the ratios cited were attached to streptavidin-coated beads (at 0.3 genes per bead).
^{b}Translation was performed in solution (non-compartmentalised) or in an emulsion (compartmentalised).
^{c}After assembly of the catalytically active metaloenzyme, PTE activity on beads was measured by paraoxon hydrolysis and the number of captured PTE molecules calculated using the published kinetic parameters of the Zn²⁺ PTE. The resulting beads were selected by enzymatic activity (FIG. 5 and text).
^{d}The % of positive events for the unsorted beads is the percentage of highly fluorescent beads in region M1 (FIG. 5, column 1); the ‘noise’ - the percentage of events in region M1 with the ΔOPD-coated beads (FIG. 5, 1a; 0.21%), was subtracted.
^{e}The ‘positive’ beads gated through R1 and M1 (FIG. 5) were sorted and reanalysed.
^{f}The genes on the sorted beads were PCR-amplified and analysed by gel electrophoresis and the ratio of OPD to ΔOPD genes after amplification was determined by densitometry (FIG. 5).
^{g}The enrichment is the final ratio of OPD:ΔOPD genes amplified from the sorted beads (column 6), divided by the starting ratio of genes coated on the beads before the selection (column 1).

TABLE II
Sequences of active PTE mutants

| | | | | | | | Rate |
|---|---|---|---|---|---|---|---|
| | | Amino Acid Residue^{a} | | | | | (relative to wild- |
| Library | Clone | 106 | 131 | 132 | 308 | 309 | type = 100%) |
| Wild-type | | I | W | F | S | Y | 100.00 |
| Library A | b5 | S | W | F | S | Y | 280.00 |
| | b3 | T | W | F | S | Y | 74.00 |
| | a9 | R | W | F | S | Y | 0.43 |
| | a10 | F | W | F | S | Y | 0.11 |
| | b2 | Y | W | F | S | Y | 0.02 |
| Library B | e3 | L | W | F | S | Y | 47.00 |
| | f6 | T | W | F | T | W | 31.00 |
| | f4 | S | W | F | L | L | 28.00 |
| | e2 | T | W | F | Q | S | 5.00 |
| | f3 | T | W | F | L | V | 2.20 |
| | e6 | I | W | F | T | C | 0.88 |
| | f9 | S | W | F | F | D | 0.29 |
| | e10 | T | W | F | S | Y | 0.10 |
| | e12 | I | W | F | T | Y | 0.02 |
| Library C | d5 | S | W | F | S | Y | 306.00 |
| | c4 | V | W | F | S | Y | 162.00 |
| | c1 | D | Y | S | S | Y | 39.00 |
| | c3 | S | W | L | S | Y | 5.70 |
| | c11 | V | W | F | S | Y | 1.80 |
| | d2 | D | R | R | S | Y | 0.52 |
| | d3 | I | Y | P | S | Y | 0.18 |
| Library D | h5 | T | W | L | S | Y | 380.00 |
| | g1 | S | W | L | M | N | 29.00 |
| | b12 | S | W | L | L | R | 6.80 |
| | h2 | T | W | F | S | Y | 5.00 |
| | h7 | Q | N | T | K | H | 4.50 |
| | g12 | C | S | T | L | N | 3.80 |
| | g3 | L | G | V | S | F | 3.30 |
| | h4 | C | W | L | E | S | 2.60 |
| | g5 | T | H | C | Q | A | 0.30 |
| | h9 | T | H | C | Q | A | 0.29 |
| | d11 | L | G | V | S | A | 0.29 |
| | b11 | S | G | W | M | T | 0.22 |
| | d9 | T | H | L | S | A | 0.16 |
| | b10 | H | G | W | L | T | 0.04 |

^{a}Residues diversified in the libraries are indicated in red, undiversified residues in black.

TABLE III
Kinetics of PTE mutants

| | | | | | | | | | | k_{cat} | K_{M} | k_{cat}/K_{M} |
|---|---|---|---|---|---|---|---|---|---|---|---|---|
| | | Amino Acid Residue^{a} | | | | | k_{cat} | K_{M} | k_{cat}/K_{M} | relative | relative | relative |
| Library | Clone | 106 | 131 | 132 | 308 | 309 | (s⁻¹) | (mM) | (M⁻¹V⁻¹ × 10⁵) | (mutant/w.t.) | (mutant/w.t.) | (mutant/w.t.) |
| Wild-type | | I | W | F | S | Y | 2280 | 0.023 ± 0.003 | 990 | 1.0 | 1.0 | 1.0 |
| Library D | h5 | T | W | L | S | Y | 279300 | 0.82 ± 0.07 | 3400 | 123.0 | 36 | 3.4 |
| | b12 | S | W | L | L | R | 7410 | 2.2 ± 0.04 | 34.0 | 3.3 | 96 | 0.034 |
| | h4 | C | W | L | E | S | 1710 | 3.3 ± 0.1 | 5.2 | 0.8 | 143 | 0.0052 |
| | g1 | S | W | L | M | N | 570 | 1.27 ± 0.13 | 4.5 | 0.3 | 55 | 0.0045 |
| | g3 | L | G | V | S | F | 513 | 1.59 ± 0.02 | 3.2 | 0.2 | 69 | 0.0033 |
| | h2 | T | W | F | S | Y | 342 | 0.41 ± 0.02 | 8.3 | 0.2 | 18 | 0.0084 |
| Library A | b5 | S | W | F | S | Y | 27930 | 0.21 ± 0.02 | 1330 | 12.0 | 9 | 1.3 |
| Library C | c1 | D | Y | S | S | Y | 22800 | 1.2 ± 0.05 | 190 | 10.0 | 52 | 0.19 |
| | d5 | S | W | F | S | Y | 13680 | 0.17 ± 0.02 | 810 | 6.0 | 7 | 0.81 |
| Library B | e3 | L | W | F | S | Y | 7980 | 0.12 ± 0.014 | 670 | 3.5 | 5 | 0.67 |

^{a}Residues diversified in the libraries are indicated in red, undiversified residues in black.

TABLE IV
Oligonucleotide Primers

| | | Annealing site |
|---|---|---|
| Name | Sequence | (FIG. 2A) |
| OPD-Flag-Bc | 5′-CATTGCCAAGCCATGGACTACAAAGATGACGATGATAAAATCACCA | |
| | ACAGCGGCGATCGGATCAATACCG-3′ | |
| OPD-HA-Fo | 5′-CGCTCCCGGGAGCTCTTATTACGCATAATCCGGCACATCATACGGA | |
| | TAACCGCCGGTACCTGACGCCCGCAAGGTCGGTGACAAGAACCG-3′ | |
| LMB2-1^{a} | 5′-CAGGCGCCATTCGCCATT-3′ | a |
| LMB2-5-biotin^{b} | 5′-CCAGCTGGCGAAAGGGGG-3′ | b |
| LMB2-6 | 5′-ATGTGCTGCAAGGCGATT-3′ | c |
| LMB2-8 | 5′-GTTTTCCCAGTCACGACG-3′ | d |
| LMB2-9 | 5′-GTAAAACGACGGCCAGT-3′ | e |
| pIV-B1^{a} | 5′-GCGTTGATGCAATTTCT-3′ | f |
| pIV-B5 | 5′-CCTGCTCGCTTCGCTAC-3′ | g |
| pIV-B6 | 5′-TTGGAGCCACTATCGAC-3′ | h |
| pIV-B8 | 5′-CACACCCGTCCTGTGGA-3′ | i |
| pIV-B9 | 5′-TATCCGGATATAGTTCC-3′ | j |
| T7 | 5′-TAATACGACTCACTATAGGG-3′ | k |
| OPDPCR-B5 | 5′-ACCAACAGCGGCGATCGGATC-3′ | l |
| OPDPCR-B6 | 5′-AATACCGTGCGCGGTCCTATC-3′ | m |
| OPDPCR-F5 | 5′-GGATGCCCAGGAGGGCTGATG-3′ | n |
| OPDPCR-F6 | 5′-CACTCGCATTATCTTCTAGAC-3′ | o |
| LibA-Fo^{c} | 5′-AAGGTTCCAACGTCTCGCGACCSNNATCGAAAGTCGACACATC-3′ | |
| LibA-Ba | 5′-AACCTTGGAACGTCTCGGTCGCGACGTCAGTTTATTGGCC-3′ | |
| LibB-Fo^{c} | 5′-AAGGTTCCAACGTCTCGGTGACSNNSNNCGAAAACCCGAACAGCCA-3′ | |
| LibB-Ba | 5′-AACCTTGGAACGTCTCGTCACCAACATCATGGAC-3′ | |
| LibC-Fo^{c} | 5′-AAGGTTCCAACGTCTCCGGGTCSNNSNNCAAGCCGGTCGCCGCCAC-3′ | |
| LibC-Ba | 5′-AACCTTGGAACGTCTCGACCGCCACTTTCGATG-3′ | |

^{a}These primers were also synthesised with a triple biotin at the 5′-end (Oswel, UK) and designated LMB2-1 tribiotin etc.
^{b}Contains a single biotin at the 5′-end.
^{c}N = A, G, C or T; S = G or C.

### REFERENCES

Amstutz, P., Forrer, P., Zahnd, C. and Pluckthun, A. (2001) In vitro display technologies: novel developments and applications. Curr Opin Biotechnol, 12, 400-405.
Anderson, C. W., Straus, J. W. and Dudock, B. S. (1983) Methods Enzymol, 101, 635-44.
Anderson, J. E. (1993) Curr. Op. Struct. Biol., 3, 24-30. Ash, M. and Ash, I. (1993) Handbook of industrial surfactants. Gower, Aldershot.
Atwell, S. and Wells, J. A. (1999) Selection for improved subtiligases by phage display. Proc Natl Acad Sci USA, 96, 9497-9502.
Baccanari, D. P., Averett, D., Briggs, C. and Burchall, J. (1977) Biochemistry, 16, 3566-72. Barany, F. (1991) PCR Methods Applic., 1, 5-16.
Baez. S., Segura-Aguilar, J., Widersten, M., Johansson, A.-S. & Mannervik, B. (1997) Biochem. J. 324, 25-28.
Bass, S., Greene, R. and Wells, J. A. (1990) Proteins, 8, 309-14.
Becher, P. (1957) Emulsions: theory and practice. Reinhold, New York. Benita, S., Ed. (1996). Microencapsulation: methods and industrial applications. Drugs and pharmaceutical sciences. Edited by Swarbrick, J. New York: Marcel Dekker.
Benner, S. A. (1994) Trends Biotechnol, 12, 158-63.
Benning, M. M., Hong, S. B., Raushel, F. M. and Holden, H. M. (2000) The binding of substrate analogs to phosphotriesterase. J Biol Chem, 275, 30556-30560.
Berman, J., Eisenberg, S. and Tye, B. K. (1987) Methods Enzymol, 155, 528-37.
Betlach, L., Hershfield, V., Chow, L., Brown, W., Goodman, H. M. & Boyer, H. W. (1976). A restriction endonuclease analysis of the bacterial plasmid controlling the EcoRI restriction and modification of DNA. Federation Proceedings 35, 2037-2043.
Blattner, F. R. and Dahlberg, J. E. (1972) Nature New Biol, 237, 227-32.
Bougueleret, L., Schwarzstein, M., Tsugita, A. & Zabeau, M. (1984). Characterization of the genes coding for the Eco RV restriction and modification system of Escherichia coli. Nucleic Acids Res 12(8), 3659-76.
Bru, R. & Walde, P. (1991). Product inhibition of alpha-chymotrypsin in reverse micelles. Eur J Biochem 199(1), 95-103. Bru, R. & Walde, P. (1993). Catalytic activity of elastase in reverse micelles. Biochem Mol Biol Int 31(4), 685-92.
Brynes, P. J., Andrade, P. & Gordon D. (1982). Sensitive fluorogenic substrates for the detection of trypsin-like proteases and pancreatic elastase. Anal. Biochem. 126, 447.
Cahill, P., Foster, K. and Mahan, D. E. (1991) Clin Chem, 37, 1482-5.
Caldwell, S. R., Newcomb, J. R., Schlecht, K. A. and Raushel, F. M. (1991) Limits of diffusion in the hydrolysis of substrates by the phosphotriesterase from Pseudomonas diminuta. Biochemistry, 30, 7438-7444.
Chakrabarti, A. C., Breaker, R. R., Joyce, G. F. & Deamer, D. W. (1994). Production of RNA by a polymerase protein encapsulated within phospholipid vesicles. J Mol Evol 39(6), 555-9.
Chamberlin, M. and Ring, J. (1973) J Biol Chem, 248, 2235-2244.
Chang, T. M. (1987). Recycling of NAD(P) by multienzyme systems immobilized by microencapsulation in artificial cells. Methods Enzymol 136(67), 67-82.
Chang, T. M. S. (1992). Recent advances in artificial cells based on microencapsulation. In Microcapsules and nanoparticles in medicine and pharmacy (Donbrow, M., ed.), pp. 323-339. CRC Press, Boca Raton, Fla.
Chapman, K. B. and Szostak, J. W. (1994) Curr. op. Struct. Biol., 4, 618-622.
Chen-Goodspeed, M., Sogorb, M. A., Wu, F. and Raushel, F. M. (2001b) Enhancement, relaxation, and reversal of the stereoselectivity for phosphotriesterase by rational evolution of active site residues. Biochemistry, 40, 1332-1339.
Chen-Goodspeed, M., Sogorb, M. A., Wu, F., Hong, S. B. and Raushel, F. M. (2001a) Structural determinants of the substrate and stereochemical specificity of phosphotriesterase. Biochemistry, 40, 1325-1331.
Chetverin, A. B. and Spirin, A. S. (1995) Prog Nucleic Acid Res Mol Biol, 51, 225-70.
Chiang, C. M. and Roeder, R. G. (1993) Expression and purification of general transcription factors by FLAG epitope-tagging and peptide elution. Pept Res, 6, 62-64.
Clackson, T. and Wells, J. A. (1994) Trends Biotechnol, 12, 173-84.
Cormack, B. P., Valdivia, R. H. & Falkow, S. (1996). FACS-optimized mutants of the green fluorescent protein (GFP). Gene(33).
Craig, D. et al. (1995). Fluorescence-based enzymatic assay by capillary electrophoresis laser-induced fluorescence detection for the determination of a few β-galactosidase molecules. Anal. Biochem. 226, 147.
Creagh, A. L., Prausnitz, J. M. & Blanch, H. W. (1993). Structural and catalytic properties of enzymes in reverse micelles. Enzyme Microb Technol 15(5), 383-92.
Cull, M. G., Miller, J. F. and Schatz, P. J. (1992) Proc Natl Acad Sci USA, 89, 1865-9.
Delagrave, S., Hawtin, R. E., Silva, C. M., Yang, M. M. & Youvan, D. C. (1995). Red-shifted excitation mutants of the green fluorescent protein. Biotechnology N Y 13(2), 151-4.
Demartis, S., Huber, A., Viti, F., Lozzi, L., Giovannoni, L., Neri, P., Winter, G. & Neri, D. (1999). J. Mol. Biol. 286, 617-633.
Dickinson, E. (1994) In Wedlock, D. J. (ed.), Emulsions and droplet size control. Butterworth-Heine-mann, Oxford, Vol. pp. 191-257.
Dumas, D. P., Caldwell, S. R., Wild, J. R. and Raushel, F. M. (1989) Purification and properties of the phosphotriesterase from Pseudomonas diminuta. J Biol Chem, 264, 19659-19665.
Ehrig, T., O'Kane, D. J. & Prendergast, F. G. (1995). Green-fluorescent protein mutants with altered fluorescence excitation spectra. Febs Lett 367(2), 163-6.
Ellington, A. D. and Szostak, J. W. (1990) Nature, 346, 81822.

Ellman, J., Mendel, D., Anthony, C. S., Noren, C. J. and Schultz, P. G. (1991) Methods Enzymol, 202, 301-36.
Fahy, E., Kwoh, D. Y. and Gingeras, T. R. (1991) PCR Methods Appl, 1, 25-33.
Fastrez, J. (1997) In vivo versus in vitro screening or selection for catalytic activity in enzymes and abzymes. Mol Biotechnol, 7, 37-55.
Fersht, A. (1999) Structure and mechanism in protein science. W.H. Freeman and Company, New York.
Field, J., Nikawa, J., Broek, D., MacDonald, B., Rodgers, L., Wilson, I. A., Lerner, R. A. and Wigler, M. (1988) Purification of a RAS-responsive adenylyl cyclase complex from Saccharomyces cerevisiae by use of an epitope addition method. Mol Cell Biol, 8, 2159-2165.
Fields, S. & Song, O. (1989) A novel genetic system to detect protein-protein interactions. Nature 340, 245-6.
Finch, C. A. (1993). Encapsulation and controlled release. Spec. Publ.-R. Soc. Chem. 138, 35.
Firestine, S. M., Salinas, F., Nixon, A. E., Baker, S. J. and Benkovic, S. J. (2000) Using an AraC-based three-hybrid system to detect biocatalysts in vivo. Nat Biotechnol, 18, 544-547.
Fisch, I., Kontermann, R. E., Finnem, R., Hartley, O., Soler, G. A., Griffiths, A. D. and Winter, G. (1996) ProcNatl Acad Sci USA, 93, 7761-6.
Formusek, L. and Vetvicka, V. (1986). Polymeric microspheres as diagnostic tools for cell surface marker tracing. Crit. Rev. Ther. Drug Carrier Syst. 2, 137-74
Freese, E. (1959) J. Mol. Biol., 1, 87.
Friedberg, E. C., Walker, G. C. and Siede, W. (1995) DNA repair and mutagenesis. ASM Press, Washington D.C.
Georgiou, G. (2000) Analysis of large libraries of protein mutants using flow cytometry. Adv Protein Chem, 55, 293-315.
Georgiou, G., Stathopoulos, C., Daugherty, P. S., Nayak, A. R., Iverson, B. L. and Curtiss, R., 3rd. (1997) Display of heterologous proteins on the surface of microorganisms: from the screening of combinatorial libraries to live recombinant vaccines. Nat Biotechnol, 15, 29-34.
Ghadessy, F. J., Ong, J. L. and Holliger, P. (2001) Directed evolution of polymerase function by compartmentalized self-replication. Proc Natl Acad Sci USA, 98, 4552-4557.
Gold, L., Polisky, B., Uhlenbeck, O. and Yarus, M. (1995) Annu Rev Biochem, 64, 763-97.
Green, R. and Szostak, J. W. (1992) Science, 258, 1910-5.
Gregoriadis, G. (1976) Methods Enzymol, 44, 21 8-27.
Griffiths, A. D. and Duncan, A. R. (1998) Strategies for selection of antibodies by phage display. Curr Opin Biotechnol, 9, 102-108.
Griffiths, A. D. and Tawfik, D. S. (2000) Man-made enzymes-from design to in vitro compartmentalisation. Curr Opin Biotechnol, 11, 338-353.
Griffiths, A. D., Williams, S. C., Hartley, O., Tomlinson, I. M., Waterhouse, P., Crosby, W. L., Kontermann, R. E., Jones, P. T., Low, N. M., Allison, T. J. and et a.l. (1994) Embo J, 13, 3245-60.
Guixe, V., Rodriguez, P. H. & Babul, J. (1998). Ligand-induced conformational transitions in Escherichia coli phosphofructokinase 2. Biochemistry 37, 13269-75.
Haber, J., Maslakiewicz, P., Rodakiewicz, N. J. & Walde, P. (1993). Activity and spectroscopic properties of bovine liver catalase in sodium bis(2-ethylhexyl)sulfosuccinate/isooctane reverse micelles. Eur J Biochem 217(2), 567-73.
Habig, W. H. & Jakoby, W. B. (1981). Methods in Enzymology, 77, 398-405.
Hanes, J. & Pluckthun, A. (1997). In vitro selection and evolution of functional proteins by using ribosome display. Proc. Natl. Acad. Sci. USA 94, 4937-4942.
Haugland, R. H. (ed.). (1996). Handbook of Fluorescent Probes and Research Chemicals, Sixth Edition, Molecular Probes, pp201-250.
Heim, R. & Tsien, R. Y. (1996). Engineering green fluorescent protein for improved brightness, longer wavelengths and fluorescence resonance energy transfer. Curr Biol 6(2), 178-82.
Hermanson, G. T. (1996) Bioconjugate techniques. Academic Press, San Diego.
Hochuli, E., Dobeli, H. and Schacher, A. (1987) J Chromatogr, 411, 177-84.
Hong, S. B. and Raushel, F. M. (1999) Stereochemical constraints on the substrate specificity of phosphotriesterase. Biochemistry, 38, 1159-1165.
Hong, S. B., Kuo, J. M., Mullins, L. S. and Raushel, F. M. (1995) Co2 Is Required for the Assembly of the Binuclear Metal Center of Phosphotriesterase. Journal of the American Chemical Society, 117, 7580-7581.
Hoogenboom, H. R. (1997). Designing and optimizing library selection strategies for generating high-affinity antibodies. Trends Biotechnol. 15, 62-70.
Hoogenboom, H. R., et al., (1991) Nucl. Acids Res., 91, 4133-4137.
Huang, Z. et al. (1992). A sensitive competitive ELISA for 2,4-dinitrophenol using 3,6-fluorescein diphosphate as a fluorogenic substrate. J. Immunol. Meth. 149, 261.
Janda, K. D., Lo, L.-C., Lo, C.-H. L., Sim, M., -M., Wang, R., Wong, C.-H. and Lerner, R. A. (1997) Science, 275, 945-948.
Jestin, J.-L., Kristensen, P. & Winter, G. (1999). Angew. Chem. Int. Ed. Engl. 38, 1124-1127.
Johannsson, A. (1991) In Price, C. P. and Newman, D. J. (ed.), Heterogeneous enzyme immunoassays. Stockton Press, New York, Vol. pp. 295-325.
Johannsson, A. and Bates, D. L. (1988) In Kemeny, D. M. and Challacombe, S.i. (ed.) Amplification by second enzymes. John Wiley, Chichester, Vol. pp. 85-106.
Jones, L. J. et al. (1997). Quenched BODIPY dye-labeled casein substrates for the assay of protease activity by direct fluorescence measurement. Anal. Biochem. 251, 144.
Joyce, G. F. (1994) Curr. op. Structural Biol., 4, 331-336.
Kadir, F. H. and Moore, G. R. (1990) Febs Lett, 276, 81-4.
Kallen, R. G. & Jencks, W. P. (1966). The mechanism of the condensation of formaldehyde with tetrahydrofolic acid. J. Biol. Chem. 241, 5851-5863.
Katanaev, V. L., Kurnasov, O. V. and Spirin, A. S. (1995) Febs Lett, 359, 89-92.
Keefe, A. D. and Szostak, J. W. (2001) Functional proteins from a random-sequence library. Nature, 410, 715-718.
Keij, J. F., Groenewegen, A. C. & Visser, J. W. M. (1994) High-speed photodamage cell sorting: An evaluation of the ZAPPER prototype. Methods in cell biology 42, 371-358.
Kerker, M. (1983) Elastic and inelastic light scattering in flow cytometry. Cytometry 4, 1-10
Klug, A. (1995) Ann N Y Acad Sci, 758, 143-60.
Klug, A. and Schwabe, J. W. (1995) Faseb T, 9, 597-604.
Kolb, V. A., Makeyev, E. V., Kommer, A. and Spirin, A. S. (1995) Biochem Cell Biol, 73, 1217-20.
Kowalczykowski, S. C., Dixon, D. A., Eggleston, A. K., Lauder, S. D. and Rehrauer, W. M. (1994) Microbiol Rev, 58, 401-65.
Krumdiek, C. L. & Baugh, C. M. (1980) Solid-phase synthesis of pteroylpolyglutamates. Methods Enzymol. pp. 524-529
Kumar, A., Kumar, A. & Katiyar, S. S. (1989). Activity and kinetic characteristics of glutathione reductase in vitro in reverse micellar waterpool. Biochim Biophys Acta 996(1-2), 1-6.
Landegren, U., Kaiser, R., Sanders, J. and Hood, L. (1988) Science, 241, 1077-80.
Lesley, S. A., Brow, M. A. & Burgess, R. R. (1991). Use of in vitro protein synthesis from polymerase chain reaction-generated templates to study interaction of Escherichia coli transcription factors with core RNA polymerase and for epitope mapping of monoclonal antibodies. J Biol Chem 266(4), 2632-8.
Lesley, S. A. (1995) Methods Mol Biol, 37, 265-78.
Leung, D. W., Chen, E. and Goeddel, D. V. (1989) Technique, 1, 11-15.
Liao, H., McKenzie, T. and Hageman, R. (1986) Proc Natl Acad Sci USA, 83, 576-80.
Lim, F. & Sun, A. M. (1980). Microencapsulated islets as bioartificial endocrine pancreas. Science 210(4472), 908-10.
Lim, F., Ed. (1984). Biomedical applications of microencapsulation. Boca Raton, Fla.: CRC Press.
Lissant, K. J., ed Emulsions and emulsion technology. Surfactant Science New York: Marcel Dekker, 1974.
Lissant, K. J., ed. Emulsions and emulsion technology. Surfactant Science New York: Marcel Dekker, 1984.
Lorenz, W. W., McCann, R. O., Longiaru, M. & Cormier, M. J. (1991). Isolation and expression of a cDNA encoding Renilla reniformis luciferase. Proc Natl Acad Sci USA 88(10), 4438-42.
Low, N. M., Holliger, P. H. and Winter, G. (1996) J Mol Biol, 260, 359-68.
Lowman, H. B., Bass, S. H., Simpson, N. and Wells, J. A. (1991) Biochemistry, 30, 10832-8.

Luisi, P. L. & B., S.-H. (1987). Activity and conformation of enzymes in reverse micellar solutions. Methods Enzymol 136(188), 188-216.
Ma, C., Kudlicki, W., Odom, O. W., Kramer, G. and Hardesty, B. (1993) Biochemistry, 32, 7939-45.
Mackenzie, N. M. & Pinder, A. C. (1986). The application of flow microfluorimetry to biomedical research and diagnosis: a review. Dev. Biol. Stand. 64, 181-193.
Magdassi, S., Frenkel, M., Carti, N. and Cazan, R. (1984) 97, 377-379.
Mahajan, N. P., Linder, K., Berry, G., Gordon, G. W., Heim, R. & Herman, B. (1998). Bcl-2 and Bax interactions in mitochondria probed with green fluorescent protein and fluorescence resonance energy transfer. Nat Biotechnol 16(6), 547-52.
Manley, J. L., Fire, A., Samuels, M. and Sharp, P. A. (1983) Methods Enzymol, 101, 568-82.
Mao, Q. & Walde, P. (1991). Substrate effects on the enzymatic activity of alpha-chymotrypsin in reverse micelles. Biochem Biophys Res Commun 178(3), 1105-12.
Mao, Q., Walde, P. & Luisi, P. L. (1992). Kinetic behaviour of alpha-chymotrypsin in reverse micelles. A stopped-flow study. Eur J Biochem 208(1), 165-70.
Masui, R. & Kuramitsu, S. (1998). Probing of DNA-binding sites of Escherichia coli RecA protein utilizing 1-anilinonaphthalene-8-sulfonic acid. Biochemistry 37, 12133-12143.
Matayoshi, E. D. et al. (1990). Novel fluorogenic substrates for assaying retroviral proteases by resonance energy transfer. Science 247, 954.
Mattheakis, L. C., Bhatt, R. R. and Dower, W. J. (1994) Proc Natl Acad Sci USA, 91, 9022-6.
McCafferty, J., Griffiths, A. D., Winter, G. and Chiswell, D. J. (1990) Nature, 348, 552-4.
Melton, D. A., Krieg, P. A., Rebagliati, M. R., Maniatis, T., Zinn, K. and Green, M. R. (1984) Nucleic Acids Res, 12, 703556.
Mendel, D., Cornish, V. W. and Schultz, P. G. (1995) Annu Rev Biophys Biomol Struct, 24, 435-62.
Menger, F. M. & Yamada, K. (1979). J. Am. Chem. Soc. 101, 6731-6734.
Miele, E. A., Mills, D. R. and Kramer, F. R. (1983) J Mol Biol, 171, 281-95.
Minshull, J. and Stemmer, W. P. (1999) Protein evolution by molecular breeding. Curr Opin Chem Biol, 3, 284-290.
Miroux, B., Walker, J. E. (1996) Journal of Molecular Biology, 260, 289-298
Miyawaki, A., Llopis, J., Heim, R., McCaffery, J. M., Adams, J. A., Ikura, M. & Tsien, R. Y. (1997). Fluorescent indicators for Ca2+ based on green fluorescent proteins and calmodulin. Nature 388(6645), 882-7.
Mize, P. D., Hoke, R. A., Linn, C. P., Reardon, J. E. and Schulte, T. H. (1989) Anal Biochem, 179, 229-35.
Mock, D. M., Langford, G., DuBois, D., Criscimagna, N. & Horowitz, P. (1985) Anal. Biochem. 151, 178-181.
Montigiani, S., Neri, G., Neri, P. and Neri, D. (1996) J Mol Biol, 258, 6-13.
Moore, M. J. (1995) Nature, 374, 766-7.
Mulbry, W. W. and Karns, J. S. (1989) Parathion hydrolase specified by the Flavobacterium opd gene: relationship between the gene and protein. J Bacteriol, 171, 6740-6746.
Nemoto, N., Miyamoto-Sato, E., Husimi, Y. and Yanagawa, H. (1997). In vitro virus: Bonding of mRNA bearing puromycin at the 3′-terminal end to the C-terminal end of its encoded protein on the ribosome in vitro. FEBS Letters 414, 405-408.
Ness, J. E., Del Cardayre, S. B., Minshull, J. and Stemmer, W. P. (2000) Molecular breeding: the natural approach to protein design. Adv Protein Chem, 55, 261-292.
New, R. R. C., Ed. (1990). Liposomes: a practical approach. The practical appraoch series. Edited by Rickwood, D. & Hames, B. D. Oxford: Oxford University Press.
Nissim, A., Hoogenboom, H. R. I Tomlinson, I. M., Flynn, G., Midgley, C., Lane, D. and Winter, G. (1994) Embo J, 13, 692-8.
Norman, S. O. (1980). Flow cytometry. Med. Phys. 7, 609-615.
Oberholzer, T., Albrizio, M. & Luisi, P. L. (1995a). Polymerase chain reaction in liposomes. Chemistry and Biology 2, 677-682.
Oberholzer, T., Wick, R., Luisi, P. L. & Biebricher, C. K. (1995b). Enzymatic RNA replication in self-reproducing vesicles: an approach to a minimal cell. Biochem Biophys Res Commun 207(1), 250-7.
Ogura, Y. (1955) Catalase activity at high concentrations of hydrogen peroxide. Archs. Biochem. Biophys., 57, 288-300.
Olsen, M. J., Stephens, D., Griffiths, D., Daugherty, P., Georgiou, G. and Iverson, B. L. (2000) Function-based isolation of novel enzymes from a large library. Nat Biotechnol, 18, 1071-1074.
Omburo, G. A., Kuo, J. M., Mullins, L. S. and Raushel, F. M. (1992) Journal of Biological Chemistry, 267, 13278-13283.
Parnley, S. F. and Smith, G. P. (1988) Gene, 73, 305-18.
Pederson, H., Holder, S., Sutherlin, D. P., Schwitter, U., King, D. S. & Schultz, P. G. (1998). Proc. Natl. Acad. Sci. USA 95, 10523-10528.
Pelham, H. R. and Jackson, R. J. (1976) Eur J Biochem, 67, 247-56.
Perelson, A. S. and Oster, G. F. (1979) J Theor Biol, 81, 64570.
Perez, G. M., Sanchez, F. A. & Garcia, C. F. (1992). Application of active-phase plot to the kinetic analysis of lipoxygenase in reverse micelles. Biochem J.
Petrounia, I. P. and Arnold, F. H. (2000) Designed evolution of enzymatic properties. Curr Opin Biotechnol, 11, 325-330.
Pirrung, M. C. and Huang, C. Y. (1996) A general method for the spatially defined immobilization of biomolecules on glass surfaces using “caged” biotin. Bioconjug Chem, 7, 317-321.
Pluckthun, A., Schaffitzel, C., Hanes, J. and Jermutus, L. (2000) In vitro selection and evolution of proteins. Adv Protein Chem, 55, 367-403.
Posner, B. A., Li, L., Bethell, R., Tsuji, T. and Benkovic, S. J. (1996) Biochemistry, 35, 1653-63.
Qi-X; Grabowski-GA. (1998). Acid beta-glucosidase: intrinsic fluorescence and conformational changes induced by phospholipids and saposin C. Biochemistry 37, 11544-115554.
Raushel, F. M. and Holden, H. M. (2000) Phosphotriesterase: an enzyme in search of its natural substrate. Adv Enzymol Relat Areas Mol Biol, 74, 51-93.
Roberts, B. E., Gorecki, M., Mulligan, R. C., Danna, K. J., Rozenblatt, S. and Rich, A. (1975) Proc Natl Acad Sci USA, 72, 1922-6.
Roberts, J. W. (1969) Nature, 224, 1168-74.
Roberts, R. W. & Szostak, J. W. (1997). RNA-peptide fusions for the in vitro selection of peptides and proteins. Proc. Natl. Acad. Sci. USA 94, 12297-12302.
Roberts, R. W. (1999) Totally in vitro protein selection using mRNA-protein fusions and ribosome display. Curr Opin Chem Biol, 3, 268-273.
Rolland, J. M., Dimitropoulos, K., Bishop, A., Hocking, G. R. and Nairn R. C. 1985 Fluorescence polarization assay by flow cytometry. J. Immunol. Methods 76, 1-10.
Rosenberg, M., Weissman, S. and decrombrugghe, B. (1975) J Biol Chem, 250, 4755-64.
Rosenberry, T. L. (1975) Acetylcholinesterase. Adv Enzymol Relat Areas Mol Biol, 43, 103-218.
Saiki, R. K., Gelfand, D. H., Stoffel, S., Scharf, S. J., Higuchi, R., Horn, G. T., Mullis, K. B. and Erlich, H. A. (1988) Science, 239, 487-91.
Sambrook, J., Fritsch, E. F. and Maniatis, T. (1989) Molecular cloning: a laboratory manual. Cold Spring Harbor Laboratory Press, New York.
Savage, M. D., Mattson, G., Desai, S., Nielander, G. W., Morgensen, S. and Conklin, E. J. (1994) Avidin-biotin chemistry: a handbook. Pierce Chemical Company, Rockford.
Schatz, P. J., Cull, M. G., Martin, E. L. and Gates, C. M. (1996) Screening of peptide libraries linked to lac repressor. Methods Enzymol, 267, 171-191.
Schick, M. J. (1966) Nonionic surfactants. Marcel Dekker, New York.
Shapiro, H. M. (1983). Multistation multiparameter flow cytometry: a critical review and rationale. Cytometry 3, 227-243.
Sherman, P. (1968) Emulsion science. Academic Press, London.
Sidhu, S. S. (2000) Phage display in pharmaceutical biotechnology. Curr Opin Biotechnol, 11, 610-616.
Siemering, K. R., Golbik, R., sever, R. and Haselhof, J. (1996). Mutations that suppress the thermosensitivity of green fluorescent protein. Current Biology 6, 1653-1663.
Smith, G. P. (1985) Science, 228, 1315-7.
Soumillion, P. and Fastrez, J. (2001) Novel concepts for selection of catalytic activity. Curr Opin Biotechnol, 12, 387-394.
Soumillion, P., Jespers, L., Bouchet, M., Marchand, B. J., Winter, G. and Fastrez, J. (1994) J Mol Biol, 237, 415-22.
Stemmer, W. P. (1994a) Nature, 370, 389-91.
Stemmer, W. P. (1994b) Proc Natl Acad Sci USA, 91, 10747-51.
Stofko, H. R., Carr, D. W. and Scott, J. D. (1992) Febs Lett, 302, 274-8.
Sun, A. M., Vasek, I. & Tai, I. (1992). Microencapsulation of living cells and tissues. In Microencapsulation and nanoparticles in medicine and pharmacy (Donbrow, M., ed.), pp. 315-322. CRC Press, Boca Raton, Fla.
Sundberg, S. A., Barrett, R. W., Pirrung, M., Lu, A. L., Kiangsoontra, B. and Holmes, C. P. (1995) Spatially-addressable immobilisation of macromolecules on solid supports. J. Am. Chem. Soc., 117, 12050-12057.
Tawfik, D. S., Green, B. S., Chap, R., Sela, M. & Eshhar, Z. (1993). catELISA: a facile general route to catalytic antibodies. Proc Natl Acad Sci USA 90(2), 373-7.
Tawfik, D. S. and Griffiths, A. D. (1998) Man-made cell-like compartments for molecular evolution. Nat Biotechnol, 16, 652-656.
Tawfik, D. S., Eshhar, Z., Bentolila, A. and Green, B. S. (1993a) 1,8-diabycyclo[5.4.0]undecane mediated transesterification of p-nitrophenyl phosphonates—a novel route to phosphono esters. SYNTHESIS-STUTTGART, 10, 968-972.
Tawfik, D. S., Green, B. S., Chap, R., Sela, M. and Eshhar, Z. (1993b) catELISA: a facile general route to catalytic antibodies. Proc Natl Acad Sci USA, 90, 373-377.
Tawfik, D. S., Lindner, A. B., Chap, R., Kim, S.-H., Green, B. S. and Eshhar, Z. (1997) ELISA-based detection of catalytic antibodies and enzymes. In Lefkovits, I. (ed.), Immunology Methods Manual. Academic Press, London, pp. 553-559.
Tokatlidis, K., Friguet, B., Deville, B. D., Baleux, F., Fedorov, A. N., Navon, A., Djavadi, O. L. and Goldberg, M. E. (1995) Philos Trans R Soc Lond B Biol Sci, 348, 89-95.
Tripet, B., Yu, L., Bautista, D. L., Wong, W. Y., Irvin, R. T. and Hodges, R. S. (1996) Protein Engng., 9, 1029-1042.
Tuerk, C. and Gold, L. (1990) Science, 249, 505-10.
van Hal, D. A., Bouwstra, J. A. & Junginger, H. E. (1996). Nonionic surfactant vesicles containing estradiol for topical application. In Microencapsulation: methods and industrial applications (Benita, S., ed.), pp. 329-347. Marcel Dekker, New York.
Vanhooke, J. L., Benning, M. M., Raushel, F. M. and Holden, H. M. (1996) Three-dimensional structure of the zinc-containing phosphotriesterase with the bound substrate analog diethyl 4-methylbenzylphosphonate. Biochemistry, 35, 6020-6025.
Voss, E. W. 1993. Kinetic measurements of molecular interactions by spectrofluorometry. J. Mol. Recognit. 6, 51-58.
Wahler, D. and Reymond, J. L. (2001) Novel methods for biocatalyst screening. Curr Opin Chem Biol, 5, 152-158.
Walde, P., Goto, A., Monnard, P.-A., Wessicken, M. & Luisi, P. L. (1994). Oparin's reactions revisited: enzymatic synthesis of poly(adenylic acid) in micelles and self-reproducing vesicles. J. Am. Chem. Soc. 116, 7541-7547.
Walde, P., Han, D. & Luisi, P. L. (1993). Spectroscopic and kinetic studies of lipases solubilized in reverse micelles. Biochemistry 32(15), 4029-34.
Walde, P., Peng, Q., Fadnavis, N. W., Battistel, E. & Luisi, P. L. (1988). Structure and activity of trypsin in reverse micelles. Eur J Biochem 173(2), 401-9.
Walker, G. T., Fraiser, M. S., Schram, J. L., Little, M. C., Nadeau, J. G. and Malinowski, D. P. (1992) Nucleic Acids Res, 20, 1691-6.
Wang, G. T. et al. (1990). Design and synthesis of new fluorogenic HIV protease substrates based on resonance energy transfer. Tetrahedron Lett. 31, 6493.
Weil, P. A., Luse, D. S., Segall, J. and Roeder, R. G. (1979) Cell, 18, 469-84.
Whateley, T. L. (1996). Microcapsules: preparation by interfacial polymerisation and interfacial complexation and their applications. In Microencapsulation: methods and industrial applications (Benita, S., ed.), pp. 349-375. Marcel Dekker, New York.
Wick, R. & Luisi, P. L. (1996). Enzyme-containing liposomes can endogenously produce membrane-constituting lipids. Chem Biol 3(4), 277-85.
Widersten, M. and Mannervik,. B. (1995) J Mol Biol, 250, 115-22.
Williams, J. W., Morrison, J. F. and Duggleby, R. G. (1979) Biochemistry, 18, 2567-73.
Winter, G., Griffiths, A. D., Hawkins, R. E. and Hoogenboom, H. R. (1994) Annu Rev Immunol, 12, 433-55.
Wittrup, K. D. (2001) Protein engineering by cell-surface display. Curr Opin Biotechnol, 12, 395-399.
Wyatt, J. R. 1 Chastain, M. and Puglisi, J. D. (1991) Biotechniques, 11, 764-9.
Yamagishi, J., Kawashima, H., Matsuo, N., Ohue, M., Yamayoshi, M., Fukui, T., Kotani, H., Furuta, R., Nakano, K. and Yamada, M. (1990) Protein Eng, 3, 713-9.
Yelamos, J., Klix, N., Goyenechea, B., Lozano, F., Chui, Y. L., Gonzalez, F. A., Pannell, R., Neuberger, M. S. and Milstein, C. (1995) Nature, 376, 225-9.
Zaccolo, M., Williams, D. M., Brown, D. M. and Gherardi, E. (1996) J Mol Biol, 255, 589-603.
Zakrzewski, S. F. (1980) Preparation of tritiated dihydrofolic acid of high specific activity. Methods Enzymol. pp. 539—.
Zaug, A. J. and Cech, T. R. (1986) Biochemistry, 25, 4478-82.
Zaug, A. J. and Cech, T. R. (1986) Science, 231, 470-5.
Zaug, A. J., Been, M. D. and Cech, T. R. (1986) Nature, 324, 429-33.
Zubay, G. (1973) Annu Rev Genet, 7, 267-87.
Zubay, G. (1980) Methods Enzymol, 65, 856-77.

1. A method for isolating one or more genetic elements encoding a gene product having a desired activity, comprising the steps of:
a) providing a population of genetic elements and expressing the genetic elements to produce their respective gene product(s), such that each molecule of gene product is linked to the genetic element encoding it at a ratio of one molecule of gene product per genetic element or less;
b) compartmentalising the genetic elements into microcapsules; and
c) sorting the genetic elements according to the activity of the gene product.

2. A method according to claim 1, wherein in step (b) the activity of the desired gene product within the microcapsule results, directly or indirectly, in the modification of the genetic element encoding the gene product to enable the isolation of the genetic element.

3. A method according to claim 2, wherein a part of the genetic element is a ligand and the desired gene product within the microcapsule binds, directly or indirectly, to said ligand to enable the isolation of the genetic element.

4. A method according to claim 3, wherein the ligand is also encoded by the genetic element.

5. A method according to claim 2, wherein the product of the activity of the desired gene product within the microcapsule results, directly or indirectly, in the generation of a product which is subsequently complexed with the genetic element and enables its isolation.

6. A method according to claim 1, wherein step (a) comprises:
expressing the genetic elements to produce their respective gene products within microcapsules, linking the gene products to the genetic elements encoding them and isolating the complexes thereby formed.

7. A method according to claim 6, wherein the complexes are subjected to a further compartmentalisation step in order to isolate the genetic elements encoding a gene product having the desired activity.

8. A method according to claim 1 further comprising the additional step of:
(d) introducing one or more mutations into the genetic element(s) isolated in step (c).

9. A method according to claim 1 further comprising iteratively repeating one or more of steps (a) to (d).

10. A method according to claim 1 further comprising amplifying the genetic elements.

11. A method according to claim 1, wherein microencapsulation is achieved by forming a water-in-oil emulsion of the aqueous solution in an oil-based medium.

12. A method according to claim 1, wherein the genetic element comprises the gene attached to a microbead.

13. A method according to claim 1, wherein the microbead is nonmagnetic, magnetic or paramagnetic.

14. A method according to claim 1, wherein the genetic elements or microcapsules containing them are sorted by detection of a change in their fluorescence.

15. A method according to claim 14, wherein the sorting of genetic elements or microcapsules is performed using a fluorescence activated cell sorter (FACS).

16. A method according to claim 14, wherein the different fluorescence properties of the substrate and the product are due to fluorescence resonance energy transfer (FRET).

17. A method according to claim 1, wherein the internal environment of the microcapsules is modified by the addition of one or more reagents to the oil phase.

18. A product when isolated according to the method of claim 1.

19. A product according to claim 18 which has a higher activity than an unselected equivalent.

20. A product according to claim 19, which has a higher activity than any pre-existing equivalent.

21. A product according to claim 19, which is a mutant of a hydrolase.

22. A product according to claim 21, which is a mutant of a phosphotriesterase.

23. A phosphotriesterase having a k_{cat} of 10⁵ s⁻¹ or more.

24. A phosphotriesterase having a k_{cat} of 2.8×10⁵ S⁻¹.

25. A phosphotriesterase comprising one or more of the mutations selected from the group consisting of: I106T and F132L; I106S, F132L, S308L and Y309R; I106S; I106L; and I106D, W131Y and F132S.

26. A method for preparing a gene product, comprising the steps of:
(a) preparing a genetic element encoding the gene product;
(b) compartmentalising genetic elements into microcapsules;
(c) expressing the genetic elements to produce their respective gene products within the microcapsules and linking the gene products to the genetic elements, such that each genetic element is linked to not more than one molecule of its respective gene product;
(d) sorting the genetic elements which produce the gene product(s) having the desired activity; and
(e) expressing the gene product having the desired activity.

27. A method for screening a compound or compounds capable of modulating the activity of a gene product, comprising the steps of:
(a) preparing a repertoire of genetic elements encoding gene product;
(b) compartmentalising the genetic elements into microcapsules;
(c) expressing the genetic elements to produce their respective gene products within the microcapsules and linking the gene products to the genetic elements, such that each genetic element is linked to not more than one molecule of its respective gene product;
(d) sorting the genetic elements which produce the gene product(s) having the desired activity; and
(e) contacting a gene product having the desired activity with the compound or compounds and monitoring the modulation of an activity of the gene product by the compound or compounds.

28. A method for preparing a compound or compounds comprising the steps of:
(a) providing a synthesis protocol wherein at least one step is facilitated by a polypeptide;
(b) preparing genetic elements encoding variants of the polypeptide which facilitates this step;
(c) compartmentalising the genetic elements into microcapsules;
(d) expressing the genetic elements to produce their respective gene products within the microcapsules and linking the gene products to the genetic elements, such that each genetic element is linked to not more than one molecule of its respective gene product;
(e) sorting the genetic elements which produce polypeptide gene product(s) having the desired activity; and
(f) preparing the compound or compounds using the polypeptide gene product identified in (e) to facilitate the relevant step of the synthesis.
